(19) United States
(12) Patent Application Publication
(10) Pub. No.: US 2019/0055283 A1
(43) Pub. Date: Feb. 21, 2019

(54) AZA-PEPTIDE ALDEHYDES AND KETONES

(71) Applicant: OHIO STATE INNOVATION FOUNDATION, Columbus, OH (US)

(72) Inventors: Ozlem Dogan EKICI, Dublin, OH (US); Christopher HADAD, Dublin, OH (US); Thomas CORRIGAN, Columbus, OH (US)

(21) Appl. No.: 16/080,781

(22) PCT Filed: Feb. 28, 2017
(86) PCT No.: PCT/US17/19900

§ 371 Date: Aug. 29, 2018

Related U.S. Application Data
(60) Provisional application No. 62/301,213, filed on Feb. 29, 2016.

(51) Int. Cl. C07K 5/02; A61P 35/00; C07K 7/02; A61P 25/28

(52) U.S. Cl. CPC C07K 5/02; A61P 35/00; A61K 38/07; A61P 25/28; C07K 7/02

(57) ABSTRACT

The present disclosure relates to compositions for inhibiting proteases, methods for synthesizing the compositions, and methods of using the disclosed protease inhibitors. Aspects of the invention include aza-peptide aldehyde and ketone compositions that inhibit proteases. The disclosed compounds, pharmaceutically acceptable salts, pharmaceutically acceptable derivatives, prodrugs, or combinations thereof can be used to treat disease or pathological conditions related to the activity of proteases associated with a specific disease or condition.

### CROSS REFERENCE TO RELATED APPLICATIONS

This application claims the benefit of U.S. Provisional Patent Application Ser. No. 62/301,213 filed Feb. 29, 2016, the disclosure of which is expressly incorporated herein by reference.

### FIELD

The present disclosure relates to compositions for inhibiting proteases, methods for synthesizing the compositions, and methods of using the disclosed protease inhibitors. Aspects of the invention include aza-peptide aldehyde and ketone compositions that inhibit proteases. The disclosed compounds, pharmaceutically acceptable salts, pharmaceutically acceptable derivatives, prodrugs, or combinations thereof can be used to treat disease or pathological conditions related to the activity of proteases associated with a specific disease or condition.

### BACKGROUND

Proteases are enzymes that catalyze the hydrolysis of peptide bonds in proteins, a process called proteolysis. Uncontrolled, excessive proteolysis can lead to a large variety of disease states including cancer, cardiovascular, inflammatory, neurodegenerative (Alzheimer's and Parkinson's diseases), bacterial, viral (HIV), and parasitic diseases. Excessive proteolysis can be stopped by inhibiting the appropriate proteases. Despite the large number of inhibitors that have been designed for proteases, currently only a few classes of inhibitors are specific for their target protease.

The current anticancer drug bortezomib (Velcade), a peptide boronic acid, is a protease inhibitor and is a well-established treatment for patients with multiple myeloma. Unfortunately, bortezomib also causes a series of severe side effects. One of the side effects is peripheral neuropathy stemming from excessive nerve damage, which was found to be due to bortezomib's lack of specificity, as it also inhibits HtrA2/Omi, an ATP-dependent serine protease. One other protease inhibitor, the peptide epoxyketone carfilzomib was approved by the FDA in 2012 and the orally bioavailable epoxyketone Oprozomib (ONX0912) is in Phase 1b clinical trials. Both of these compounds still exhibit peripheral neuropathies, but at a reduced level in comparison to bortezomib. The search for new and improved proteasome inhibitors with increased specificity and decreased toxicity remains a continuing challenge.

The compounds, compositions, and methods disclosed herein address these and other needs.

### SUMMARY

Disclosed herein are novel aza-peptide aldehyde and ketone compounds and compositions for inhibiting proteases. The disclosed compounds, pharmaceutically acceptable salts, pharmaceutically acceptable derivatives, prodrugs, or combinations thereof can be used to treat disease or pathological conditions related to the activity of proteases associated with a specific disease or condition (for example, neurodegenerative disease or cancer).

In one aspect, disclosed herein is a compound of the formula:

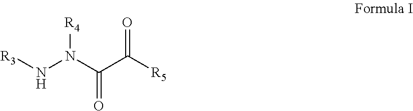

wherein R₃, R₄, and R₅ are defined herein;
or a pharmaceutically acceptable salt, derivative, hydrate or solvate thereof.

In one aspect, disclosed herein is a compound of the formula:

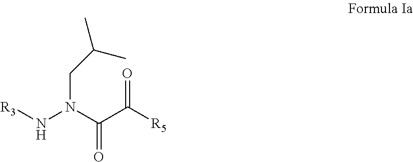

wherein R₃ and R₅ are defined herein;
or a pharmaceutically acceptable salt, derivative, hydrate or solvate thereof.

In one aspect, disclosed herein is a compound of the formula:

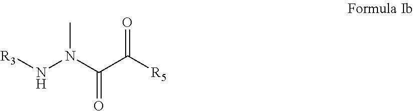

wherein R₃ and R₅ are defined herein;
or a pharmaceutically acceptable salt, derivative, hydrate or solvate thereof.

In one aspect, disclosed herein is a compound of the formula:

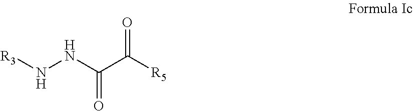

wherein R₃ and R₅ are defined herein;
or a pharmaceutically acceptable salt, derivative, hydrate or solvate thereof.

In one aspect, disclosed herein is a compound of the formula:

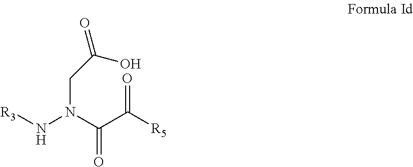

wherein R₃ and R₅ are defined herein;
or a pharmaceutically acceptable salt, derivative, hydrate or solvate thereof.

In one aspect, disclosed herein is a compound of the formula:

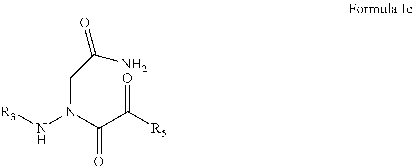

wherein R₃ and R₅ are defined herein;
or a pharmaceutically acceptable salt, derivative, hydrate or solvate thereof.

In one aspect, disclosed herein is a compound of the formula:

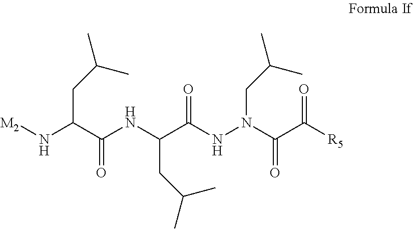

wherein M₂ and R₅ are defined herein;
or a pharmaceutically acceptable salt, derivative, hydrate or solvate thereof.

In one aspect, provided herein is a method of inhibiting a protease comprising contacting the protease with a compound of Formula I (or Formula Ia, Formula Ib, Formula Ic, Formula Id, Formula Ie, or Formula If), or a pharmaceutically acceptable salt, derivative, hydrate or solvate thereof.

In one embodiment, the protease comprises a cysteine protease. In one embodiment, the protease comprises a threonine protease. In one embodiment, the protease is caspase 3. In one embodiment, the protease is legumain. In one embodiment, the protease is MALT1. In one embodiment, the compound comprises a proteasome inhibitor.

In one aspect, provided herein is a method of treating a neurodegenerative disease in a subject in need thereof, comprising administering to the subject a therapeutically effective amount of a compound of Formula I (or Formula Ia, Formula Ib, Formula Ic, Formula Id, Formula Ie, or Formula If), or a pharmaceutically acceptable salt, derivative, hydrate or solvate thereof.

In one embodiment, the neurodegenerative disease is selected from stroke, Alzheimer's disease, Huntington's disease, Parkinson's disease, amyotrophic lateral sclerosis (ALS), multiple sclerosis (MS), and spinal muscular atrophy. In one embodiment, the neurodegenerative disease is Alzheimer's disease. In one embodiment, the neurodegenerative disease is Parkinson's disease.

In one aspect, disclosed herein is a method of treating a cancer in a subject in need thereof, comprising administering to the subject a therapeutically effective amount of a compound of Formula I (or Formula Ia, Formula Ib, Formula Ic, Formula Id, Formula Ie, or Formula If), or a pharmaceutically acceptable salt, derivative, hydrate or solvate thereof.

### DETAILED DESCRIPTION

Disclosed herein are novel aza-peptide aldehyde and ketone compounds and compositions for inhibiting proteases. The disclosed compounds, pharmaceutically acceptable salts, pharmaceutically acceptable derivatives, prodrugs, or combinations thereof can be used to treat disease or pathological conditions related to the activity of proteases associated with a specific disease or condition (for example, neurodegenerative disease or cancer).

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this invention belongs. The following definitions are provided for the full understanding of terms used in this specification.

### Terminology

As used in the specification and claims, the singular form “a,” “an,” and “the” include plural references unless the context clearly dictates otherwise. For example, the term “a cell” includes a plurality of cells, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

As used herein, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage.

As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount.” However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject.

The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

As used herein, the term “mixture” can include solutions in which the components of the mixture are completely miscible, as well as suspensions and emulsions, in which the components of the mixture are not completely miscible.

As used herein, the term “subject” or “host” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human.

### Chemical Terminology

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” as used herein refers to a non-aromatic hydrocarbon group and includes branched and unbranched, alkyl, alkenyl, or alkynyl groups.

The term “alkyl” as used herein is a branched or unbranched saturated hydrocarbon group. In some embodiments, the alkyl comprises 1 to 10 carbon atoms, such as methyl, ethyl, n-propyl, isopropyl, n-butyl, isobutyl, t-butyl, pentyl, hexyl, heptyl, octyl, nonyl, decyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like. This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkoxy” as used herein is an alkyl group bound through a single, terminal ether linkage; that is, an “alkoxy” group can be defined as —OZ¹ where Z¹ is alkyl as defined above.

The term “alkenyl” as used herein is a hydrocarbon group of from 2 to 24 carbon atoms with a structural formula containing at least one carbon-carbon double bond. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl or heteroaryl group can be substituted or unsubstituted. The aryl or heteroaryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula —C(O)H. Throughout this specification “C(O)” or “CO” is a short hand notation for C═O.

The terms “amine” or “amino” as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “carboxylic acid” as used herein is represented by the formula —C(O)OH. A “carboxylate” or “carboxyl” group as used herein is represented by the formula —C(O)O⁻.

The term “ester” as used herein is represented by the formula —OC(O)Z¹ or —C(O)OZ¹, where Z¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “acyl” refers to an alkyl group having an attached carbonyl group.

The term “halide” or “halogen” as used herein refers to the fluorine, chlorine, bromine, and iodine.

The term “hydroxyl” as used herein is represented by the formula —OH.

The term “nitro” as used herein is represented by the formula —NO₂.

The term “silyl” as used herein is represented by the formula —SiZ¹Z²Z³, where Z¹, Z², and Z³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfonyl” is used herein to refer to the sulfo-oxo group represented by the formula —S(O)₂Z¹, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

The term “thiol” as used herein is represented by the formula —SH.

The term “thio” as used herein is represented by the formula —S—. “R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxyl group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

The following abbreviations have also been used: AAla, aza-alanine residue; AAsp, aza-aspartic acid residue; AAsn, aza-asparagine; AGly, aza-glycine residue; ALeu, aza-leucine; AArg, aza-arginine residue; Bn, benzyl; Cbz, carboxybenzyl (Ph-CH₂OCO—); Ph, phenyl; Me, methyl; Et, Ethyl.

Compounds

Disclosed herein are novel aza-peptide aldehydes and ketones according to the formula:

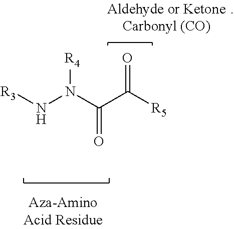

In one aspect, disclosed herein is a compound of the formula:

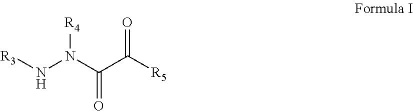

wherein R₃ is selected from the group consisting of M₁, M₂-AA₁, M₂-AA₂-AA₁, and M₂-AA₃-AA₂-AA₁;
M₁ is selected from the group consisting of NH₂—CO—, NH₂—CS—, NH₂—SO₂—, X—NH—CO—, X₂N—CO—, X—NH—CS—, X₂N—CS—, X—NH—SO₂—, X₂N—SO₂—, X—CO—, X—CS—, Y—SO₂—, Y—O—CO—, Y—O—CS—, phenyl substituted with K, phenyl disubstituted with K, and morpholine-CO—;
M₂ is selected from the group consisting of H, NH₂—CO—, NH₂—CS—, NH₂—SO₂—, X—NH—CO—, X₂N—CO—, X—NH—CS—, X₂N—CS—, X—NH—SO₂—, X₂N—SO₂—, X—CO—, X—CS—, Y—SO₂—, Y—O—CO—, Y—O—CS—, phenyl, phenyl substituted with K, phenyl disubstituted with K, and morpholine-CO—;
X is selected from the group consisting of H, C₁₋₁₀ alkyl, C₃₋₁₅ cyclized alkyl, C₁₋₁₀ fluoroalkyl, C₁₋₁₀ alkyl substituted with J, C₁₋₁₀ fluoroalkyl substituted with J, 1-admantyl, 9-fluorenyl, aryl, heteroaryl, phenyl, phenyl substituted with K, phenyl disubstituted with K, phenyl trisubstituted with K, naphthyl, naphthyl substituted with K, naphthyl disubstituted with K, naphthyl trisubstituted with K, C₁₋₁₀ fluoroalkyl with an attached phenyl group, C₁₋₁₀ alkyl with an attached phenyl group, C₁₋₁₀ alkyl with two attached phenyl groups, C₁₋₁₀ alkyl with an attached phenyl group substituted with K, C₁₋₁₀ alkyl with two attached phenyl groups substituted with K, C₁₋₁₀ alkyl with an attached naphthyl group, C₁₋₁₀ alkyl with an attached naphthyl group substituted with K, C₁₋₁₀ alkyl with an attached phenoxy group, biotinyl, and C₁₋₁₀ alkyl with an attached phenoxy group substituted with K on the phenoxy group;
Y is selected from the group consisting of C₁₋₁₀ alkyl, C₃₋₁₅ cyclized alkyl, C₁₋₁₀ fluoroalkyl, C₁₋₁₀ alkyl substituted with J, C₁₋₁₀ fluoroalkyl substituted with J, 1-admantyl, 9-fluorenyl, phenyl, phenyl substituted with K, phenyl disubstituted with K, phenyl trisubstituted with K, naphthyl, naphthyl substituted with K, naphthyl disubstituted with K, naphthyl trisubstituted with K, C₁₋₁₀ fluoroalkyl with an attached phenyl group, C₁₋₁₀ alkyl with an attached phenyl group, C₁₋₁₀ alkyl with two attached phenyl groups, C₁₋₁₀ alkyl with an attached phenyl group substituted with K, C₁₋₁₀ alkyl with two attached phenyl groups substituted with K, C₁₋₁₀ alkyl with an attached naphthyl group, C₁₋₁₀ alkyl with an attached naphthyl group substituted with K, C₁₋₁₀ alkyl with an attached phenoxy group, biotinyl, and C₁₋₁₀ alkyl with an attached phenoxy group substituted with K on the phenoxy group;
J is selected from the group consisting of halogen, CO₂H, OH, CN, NO₂, NH₂, C₁₋₁₀ alkoxy, C₁₋₁₀ alkylamino, C₂₋₁₂ dialkylamino, C₁₋₁₀ alkyl-O—CO—, C₁₋₁₀ alkyl-O—CO—NH—, and C₁₋₁₀ alkyl-S—;
K is selected from the group consisting of halogen, C₁₋₁₀ alkyl, C₁₋₁₀ perfluoroalkyl, C₁₋₁₀ alkoxy, phenoxy, NO₂, CN, OH, CO₂H, amino, C₁₋₁₀ alkylamino, C₂₋₁₂ dialkylamino, C₁₋₁₀ acyl, and C₁₋₁₀ alkoxy-CO—, and C₁₋₁₀ alkyl-S—;
AA₁, AA₂, and AA₃ are side chain blocked or unblocked amino acids with the L configuration, D configuration, or no chirality at the α-carbon independently selected from the group consisting of alanine, valine, leucine, isoleucine, proline, methionine, methionine sulfoxide, phenylalanine, tryptophan, glycine, serine, threonine, cysteine, tyrosine, asparagine, glutamine, aspartic acid, glutamic acid, lysine, arginine, histidine, phenylglycine, beta-alanine, norleucine, norvaline, alpha-aminobutanoic acid, epsilon-aminocaproic acid, citrulline, hydroxyproline, ornithine, homoarginine, sarcosine, indoline 2-carboxylic acid, 2-azetidinecarboxylic acid, pipecolinic acid (2-piperidine carboxylic acid), O-methylserine, O-ethylserine, S-methylcysteine, S-ethylcysteine, S-benzylcysteine, NH₂—CH(CH₂CHEt₂)-CO₂H, alpha-aminoheptanoic acid, NH₂—CH(CH₂₋₁-naphthyl)-CO₂H, NH₂—CH(CH₂-2-naphthyl)-CO₂H, NH₂—CH(CH₂-cyclohexyl)-CO₂H, NH₂—CH(CH₂-cyclopentyl)-CO₂H, NH₂—CH(CH₂-cyclobutyl)-CO₂H, NH₂—CH(CH₂-cyclopropyl)-CO₂H, trifluoroleucine, 4-fluorophenylalanine, lysine substituted on the epsilon nitrogen with a biotinyl group, and hexafluoroleucine;
R₄ is selected from the group consisting of hydrogen, C₁₋₁₀ alkyl, C₁₋₁₀ alkyl substituted with Q, C₁₋₁₀ alkyl substituted with phenyl, C₁₋₁₀ alkyl with an attached phenyl substituted with K, C₁₋₁₀ alkyl substituted with naphthyl, C₁₋₁₀ alkyl with an attached naphthyl substituted with K, phenyl, phenyl substituted with K, naphthyl, naphthyl substituted with K, C₁₋₁₀ alkyl substituted with CONH₂, C₁₋₁₀ alkyl substituted with CONHR₆, C₁₋₁₀ alkyl substituted with CO₂H, C₁₋₁₀ alkyl substituted with CO₂R₆, CH₂CH₂SCH₃, CH₂₋₃-indolyl, CH₂-2-thienyl, CH₂-2-furyl, CH₂-3-furyl, CH₂-2-imidazyl, C₁₋₁₀ alkyl substituted with G, C₁₋₁₀ alkyl with an attached phenyl substituted with G, C₁₋₁₀ alkyl with an attached naphthyl substituted with G, phenyl substituted with G, and naphthyl substituted with G;
R₆ is selected from the group consisting of C₁₋₁₀ alkyl and C₁₋₁₀ alkyl substituted with phenyl;
Q is selected independently from the group consisting of C₁₋₁₀ alkoxy, C₁₋₁₀ alkyl-S—, C₁₋₁₀ alkoxy substituted with phenyl, and C₁₋₁₀ alkyl-S— substituted with phenyl; G is selected independently from the group consisting of amidino (—C(═NH)NH₂), guanidino (—NHC(═NH)NH₂), isothiureido (—S—C(═NH)NH₂), amino, C₁₋₆ alkylamino, C₂₋₁₂ dialkylamino, and imidazyl;
R₅ is selected independently from the group consisting of hydrogen, R₇, NHR₈, NR₈R₉, and -AA₄-T;
R₇ is selected independently from the group consisting of C₁₋₁₀ alkyl, C₃₋₁₅ cyclized alkyl, C₁₋₁₀ alkyl with a phenyl group attached to the C₁₋₁₀ alkyl, C₃₋₁₅ cyclized alkyl with an attached phenyl group, C₁₋₁₀ alkyl with an attached phenyl group substituted with K, C₁₋₁₀ alkyl with an attached phenyl group disubstituted with K, C₁₋₁₀ alkyl with an attached phenyl group trisubstituted with K, C₃₋₁₅ cyclized alkyl with an attached phenyl group substituted with K, C₁₋₁₀ alkyl with a naphthyl group attached to the C₁₋₁₀ alkyl, C₃₋₁₅ cyclized alkyl with an attached naphthyl group, C₁₋₁₀ alkyl with an attached naphthyl group substituted with K, C₁₋₁₀ alkyl with an attached naphthyl group disubstituted with K, C₁₋₁₀ alkyl with an attached naphthyl group trisubstituted with K, and C₃₋₁₅ cyclized alkyl with an attached naphthyl group substituted with K;
T is selected independently from the group consisting of OH, OR₁₀, NHR₁₁, and NR10R11;
AA₄ is a side chain blocked or unblocked amino acid with the L configuration, D configuration, or no chirality at the α-carbon selected from the group consisting of alanine, valine, leucine, isoleucine, proline, methionine, methionine sulfoxide, phenylalanine, tryptophan, glycine, serine, threonine, cysteine, tyrosine, asparagine, glutamine, aspartic acid, glutamic acid, lysine, arginine, histidine, phenylglycine, beta-alanine, norleucine, norvaline, alpha-aminobutanoic acid, epsilon-aminocaproic acid, citrulline, hydroxyproline, ornithine, homoarginine, sarcosine, indoline 2-carboxylic acid, 2-azetidinecarboxylic acid, pipecolinic acid (2-piperidine carboxylic acid), O-methylserine, O-ethylserine, S-methylcysteine, S-ethylcysteine, S-benzylcysteine, NH₂—CH(CH₂CHEt₂)-CO₂H, alpha-aminoheptanoic acid, NH₂—CH(CH₂-1-naphthyl)-CO₂H, NH₂—CH(CH₂-2-naphthyl)-CO₂H, NH₂—CH(CH₂-cyclohexyl)-CO₂H, NH₂—CH(CH₂-cyclopentyl)-CO₂H, NH₂—CH(CH₂-cyclobutyl)-CO₂H, NH₂—CH(CH₂-cyclopropyl)-CO₂H, trifluoroleucine, 4-fluorophenylalanine, lysine substituted on the epsilon nitrogen with a biotinyl group, and hexafluoroleucine;
R₈ and R₉ are selected independently from the group consisting of H, C₁₋₁₀ alkyl, C₃₋₂₀ cyclized alkyl, C₁₋₁₀ alkyl with a phenyl group attached to the C₁₋₁₀ alkyl, C₁₋₁₀ alkyl with two phenyl groups attached to the C₁₋₁₀ alkyl, C₃₋₂₀ cyclized alkyl with an attached phenyl group, phenyl, phenyl substituted with K, C₁₋₁₀ alkyl with an attached phenyl group substituted with K, C₁₋₁₀ alkyl with an attached phenyl group disubstituted with K, C₁₋₁₀ alkyl with an attached phenyl group trisubstituted with K, C₁₋₁₀ alkyl with two phenyl groups attached to the C₁₋₁₀ alkyl and substituted with K on the phenyl group, C₁₋₁₀ alkyl with two phenyl groups attached to the C₁₋₁₀ alkyl and disubstituted with K on the phenyl groups, C₃₋₂₀ cyclized alkyl with an attached phenyl group substituted with K, C₁₋₁₀ alkyl with a morpholine [—N(CH₂CH₂)O] ring attached through nitrogen to the alkyl, C₁₋₁₀ alkyl with a piperidine ring attached through nitrogen to the alkyl, C₁₋₁₀ alkyl with a pyrrolidine ring attached through nitrogen to the alkyl, C₁₋₂₀ alkyl with an OH group attached to the alkyl, —CH₂CH₂CH₂OCH₃, C₁₋₁₀ alkyl with an attached 4-pyridyl group, C₁₋₁₀ alkyl with an attached 3-pyridyl group, C₁₋₁₀ alkyl with an attached 2-pyridyl group, C₁₋₁₀ alkyl with an attached cyclohexyl group, —NH—CH₂CH₂-(4-hydroxyphenyl), —NH—CH₂CH₂-(3-indolyl), C₁₋₁₀ alkyl with an attached 2-furyl group, C₁₋₁₀ alkyl with an attached 3-furyl group, and C₁₋₅ alkyl with an attached phenyl and a hydroxyl attached to the C₁₋₅ alkyl;
R₁₀ and R₁₁ are selected independently from the group consisting of H, C₁₋₁₀ alkyl, phenyl, nitrophenyl, and C₁₋₁₀ alkyl substituted with phenyl;
or a pharmaceutically acceptable salt, derivative, hydrate or solvate thereof.

In one aspect, disclosed herein is a compound of the formula:

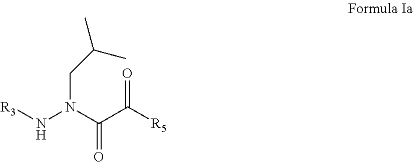

wherein R₃ and R₅ are defined herein;
or a pharmaceutically acceptable salt, derivative, hydrate or solvate thereof.

In one aspect, disclosed herein is a compound of the formula:

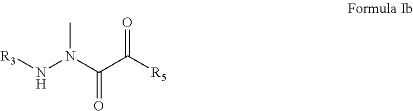

wherein R₃ and R₅ are defined herein;
or a pharmaceutically acceptable salt, derivative, hydrate or solvate thereof.

In one aspect, disclosed herein is a compound of the formula:

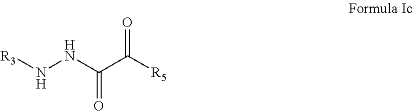

wherein R₃ and R₅ are defined herein;
or a pharmaceutically acceptable salt, derivative, hydrate or solvate thereof.

In one aspect, disclosed herein is a compound of the formula:

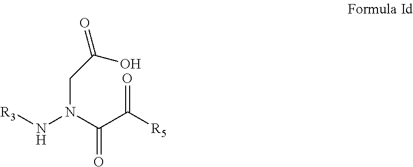

wherein R₃ and R₅ are defined herein;
or a pharmaceutically acceptable salt, derivative, hydrate or solvate thereof.

In one aspect, disclosed herein is a compound of the formula:

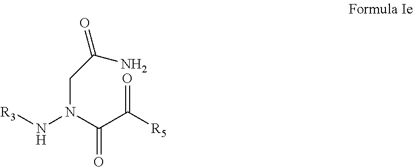

wherein R₃ and R₅ are defined herein;
or a pharmaceutically acceptable salt, derivative, hydrate or solvate thereof.

In one aspect, disclosed herein is a compound of the formula:

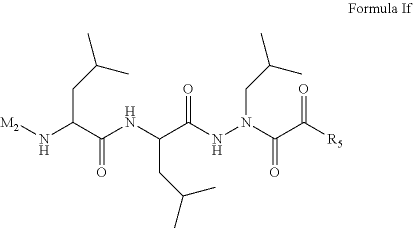

wherein M₂ and R₅ are defined herein;
or a pharmaceutically acceptable salt, derivative, hydrate or solvate thereof.

In one embodiment, R₃ is selected from the group consisting of M₁, M₂-AA₁, M₂-AA₂-AA₁, and M₂-AA₃-AA₂-AA₁.

In one embodiment, M₁ is selected from the group consisting of NH₂—CO—, NH₂—CS—, NH₂—SO₂—, X—NH—CO—, X₂N—CO—, X—NH—CS—, X₂N—CS—, X—NH—S02-, X₂N—SO₂—, X—CO—, X—CS—, Y—SO₂—, Y—O—CO—, Y—O—CS—, phenyl substituted with K, phenyl disubstituted with K, and morpholine-CO—.

In one embodiment, M₂ is selected from the group consisting of H, NH₂—CO—, NH₂—CS—, NH₂—SO₂—, X—NH—CO—, X₂N—CO—, X—NH—CS—, X₂N—CS—, X—NH—S02-, X₂N—SO₂—, X—CO—, X—CS—, Y—SO₂—, Y—O—CO—, Y—O—CS—, phenyl, phenyl substituted with K, phenyl disubstituted with K, and morpholine-CO—.

In one embodiment, X is selected from the group consisting of H, C₁₋₁₀ alkyl, C₃₋₁₅ cyclized alkyl, C₁₋₁₀ fluoroalkyl, C₁₋₁₀ alkyl substituted with J, C₁₋₁₀ fluoroalkyl substituted with J, 1-admantyl, 9-fluorenyl, aryl, heteroaryl, phenyl, phenyl substituted with K, phenyl disubstituted with K, phenyl trisubstituted with K, naphthyl, naphthyl substituted with K, naphthyl disubstituted with K, naphthyl trisubstituted with K, C₁₋₁₀ fluoroalkyl with an attached phenyl group, C₁₋₁₀ alkyl with an attached phenyl group, C₁₋₁₀ alkyl with two attached phenyl groups, C₁₋₁₀ alkyl with an attached phenyl group substituted with K, C₁₋₁₀ alkyl with two attached phenyl groups substituted with K, C₁₋₁₀ alkyl with an attached naphthyl group, C₁₋₁₀ alkyl with an attached naphthyl group substituted with K, C₁₋₁₀ alkyl with an attached phenoxy group, biotinyl, and C₁₋₁₀ alkyl with an attached phenoxy group substituted with K on the phenoxy group.

In one embodiment, Y is selected from the group consisting of C₁₋₁₀ alkyl, C₃₋₁₅ cyclized alkyl, C₁₋₁₀ fluoroalkyl, C₁₋₁₀ alkyl substituted with J, C₁₋₁₀ fluoroalkyl substituted with J, 1-admantyl, 9-fluorenyl, phenyl, phenyl substituted with K, phenyl disubstituted with K, phenyl trisubstituted with K, naphthyl, naphthyl substituted with K, naphthyl disubstituted with K, naphthyl trisubstituted with K, C₁₋₁₀ fluoroalkyl with an attached phenyl group, C₁₋₁₀ alkyl with an attached phenyl group, C₁₋₁₀ alkyl with two attached phenyl groups, C₁₋₁₀ alkyl with an attached phenyl group substituted with K, C₁₋₁₀ alkyl with two attached phenyl groups substituted with K, C₁₋₁₀ alkyl with an attached naphthyl group, C₁₋₁₀ alkyl with an attached naphthyl group substituted with K, C₁₋₁₀ alkyl with an attached phenoxy group, biotinyl, and C₁₋₁₀ alkyl with an attached phenoxy group substituted with K on the phenoxy group.

In one embodiment, J is selected from the group consisting of halogen, CO₂H, OH, CN, NO₂, NH₂, C₁₋₁₀ alkoxy, C₁₋₁₀ alkylamino, C₂₋₁₂ dialkylamino, C₁₋₁₀ alkyl-O—CO—, C₁₋₁₀ alkyl-O—CO—NH—, and C₁₋₁₀ alkyl-S—.

In one embodiment, K is selected from the group consisting of halogen, C₁₋₁₀ alkyl, C₁₋₁₀ perfluoroalkyl, C₁₋₁₀ alkoxy, phenoxy, NO₂, CN, OH, CO₂H, amino, C₁₋₀ alkylamino, C₂₋₁₂ dialkylamino, C₁₋₁₀ acyl, and C₁₋₁₀ alkoxy-CO—, and C₁₋₁₀ alkyl-S—.

In one embodiment, AA₁, AA₂, and AA₃ are side chain blocked or unblocked amino acids with the L configuration, D configuration, or no chirality at the α-carbon independently selected from the group consisting of alanine, valine, leucine, isoleucine, proline, methionine, methionine sulfoxide, phenylalanine, tryptophan, glycine, serine, threonine, cysteine, tyrosine, asparagine, glutamine, aspartic acid, glutamic acid, lysine, arginine, histidine, phenylglycine, beta-alanine, norleucine, norvaline, alpha-aminobutanoic acid, epsilon-aminocaproic acid, citrulline, hydroxyproline, ornithine, homoarginine, sarcosine, indoline 2-carboxylic acid, 2-azetidinecarboxylic acid, pipecolinic acid (2-piperidine carboxylic acid), O-methylserine, O-ethylserine, S-methylcysteine, S-ethylcysteine, S-benzylcysteine, NH₂—CH(CH₂CHEt₂)-CO₂H, alpha-aminoheptanoic acid, NH₂—CH(CH₂-1-naphthyl)-CO₂H, NH₂—CH(CH₂-2-naphthyl)-CO₂H, NH₂—CH(CH₂-cyclohexyl)-CO₂H, NH₂—CH(CH₂-cyclopentyl)-CO₂H, NH₂—CH(CH₂-cyclobutyl)-CO₂H, NH₂—CH(CH₂-cyclopropyl)-CO₂H, trifluoroleucine, 4-fluorophenylalanine, lysine substituted on the epsilon nitrogen with a biotinyl group, and hexafluoroleucine.

In one embodiment, R₄ is selected from the group consisting of hydrogen, C₁₋₁₀ alkyl, C₁₋₁₀ alkyl substituted with Q, C₁₋₁₀ alkyl substituted with phenyl, C₁₋₁₀ alkyl with an attached phenyl substituted with K, C₁₋₁₀ alkyl substituted with naphthyl, C₁₋₁₀ alkyl with an attached naphthyl substituted with K, phenyl, phenyl substituted with K, naphthyl, naphthyl substituted with K, C₁₋₁₀ alkyl substituted with CONH₂, C₁₋₁₀ alkyl substituted with CONHR₆, C₁₋₁₀ alkyl substituted with CO₂H, C₁₋₁₀ alkyl substituted with CO₂R₆, CH₂CH₂SCH₃, CH₂₋₃-indolyl, CH₂-2-thienyl, CH₂-2-furyl, CH₂-3-furyl, CH₂-2-imidazyl, C₁₋₁₀ alkyl substituted with G, C₁₋₁₀ alkyl with an attached phenyl substituted with G, C₁₋₁₀ alkyl with an attached naphthyl substituted with G, phenyl substituted with G, and naphthyl substituted with G.

In one embodiment, R₆ is selected from the group consisting of C₁₋₁₀ alkyl and C₁₋₁₀ alkyl substituted with phenyl.

In one embodiment, Q is selected independently from the group consisting of C₁₋₁₀ alkoxy, C₁₋₁₀ alkyl-S—, C₁₋₁₀ alkoxy substituted with phenyl, and C₁₋₁₀ alkyl-S— substituted with phenyl.

In one embodiment, G is selected independently from the group consisting of amidino (—C(═NH)NH₂), guanidino (—NHC(═NH)NH₂), isothiureido (—S—C(═NH)NH₂), amino, C₁-6 alkylamino, C₂₋₁₂ dialkylamino, and imidazyl.

In one embodiment, R₅ is selected independently from the group consisting of hydrogen, R₇, NHR₈, NR₈R₉, and -AA₄-T.

In one embodiment, R₇ is selected independently from the group consisting of C₁₋₁₀ alkyl, C₃₋₁₅ cyclized alkyl, C₁₋₁₀ alkyl with a phenyl group attached to the C₁₋₁₀ alkyl, C₃₋₁₅ cyclized alkyl with an attached phenyl group, C₁₋₁₀ alkyl with an attached phenyl group substituted with K, C₁₋₁₀ alkyl with an attached phenyl group disubstituted with K, C₁₋₁₀ alkyl with an attached phenyl group trisubstituted with K, C₃₋₁₅ cyclized alkyl with an attached phenyl group substituted with K, C₁₋₁₀ alkyl with a naphthyl group attached to the C₁₋₁₀ alkyl, C₃₋₁₅ cyclized alkyl with an attached naphthyl group, C₁₋₁₀ alkyl with an attached naphthyl group substituted with K, C₁₋₁₀ alkyl with an attached naphthyl group disubstituted with K, C₁₋₁₀ alkyl with an attached naphthyl group trisubstituted with K, and C₃₋₁₅ cyclized alkyl with an attached naphthyl group substituted with K.

In one embodiment, T is selected independently from the group consisting of OH, OR₁₀, NHR₁₁, and NR₁₀R₁₁.

In one embodiment, AA₄ is a side chain blocked or unblocked amino acid with the L configuration, D configuration, or no chirality at the α-carbon selected from the group consisting of alanine, valine, leucine, isoleucine, proline, methionine, methionine sulfoxide, phenylalanine, tryptophan, glycine, serine, threonine, cysteine, tyrosine, asparagine, glutamine, aspartic acid, glutamic acid, lysine, arginine, histidine, phenylglycine, beta-alanine, norleucine, norvaline, alpha-aminobutanoic acid, epsilon-aminocaproic acid, citrulline, hydroxyproline, ornithine, homoarginine, sarcosine, indoline 2-carboxylic acid, 2-azetidinecarboxylic acid, pipecolinic acid (2-piperidine carboxylic acid), O-methylserine, O-ethylserine, S-methylcysteine, S-ethylcysteine, S-benzylcysteine, NH₂—CH(CH₂CHEt₂)-CO₂H, alpha-aminoheptanoic acid, NH₂—CH(CH₂-1-naphthyl)-CO₂H, NH₂—CH(CH₂-2-naphthyl)-CO₂H, NH₂—CH(CH₂-cyclohexyl)-CO₂H, NH₂—CH(CH₂-cyclopentyl)-CO₂H, NH₂—CH(CH₂-cyclobutyl)-CO₂H, NH₂—CH(CH₂-cyclopropyl)-CO₂H, trifluoroleucine, 4-fluorophenylalanine, lysine substituted on the epsilon nitrogen with a biotinyl group, and hexafluoroleucine.

In one embodiment, R₈ and R₉ are selected independently from the group consisting of H, C₁₋₁₀ alkyl, C₃₋₂₀ cyclized alkyl, C₁₋₁₀ alkyl with a phenyl group attached to the C₁₋₁₀ alkyl, C₁₋₁₀ alkyl with two phenyl groups attached to the C₁₋₁₀ alkyl, C₃₋₂₀ cyclized alkyl with an attached phenyl group, phenyl, phenyl substituted with K, C₁₋₁₀ alkyl with an attached phenyl group substituted with K, C₁₋₁₀ alkyl with an attached phenyl group disubstituted with K, C₁₋₁₀ alkyl with an attached phenyl group trisubstituted with K, C₁₋₁₀ alkyl with two phenyl groups attached to the C₁₋₁₀ alkyl and substituted with K on the phenyl group, C₁₋₁₀ alkyl with two phenyl groups attached to the C₁₋₁₀ alkyl and disubstituted with K on the phenyl groups, C₃₋₂₀ cyclized alkyl with an attached phenyl group substituted with K, C₁₋₁₀ alkyl with a morpholine [—N(CH₂CH₂)O] ring attached through nitrogen to the alkyl, C₁₋₁₀ alkyl with a piperidine ring attached through nitrogen to the alkyl, C₁₋₁₀ alkyl with a pyrrolidine ring attached through nitrogen to the alkyl, C₁₋₂₀ alkyl with an OH group attached to the alkyl, —CH₂CH₂CH₂OCH₃, C₁₋₁₀ alkyl with an attached 4-pyridyl group, C₁₋₁₀ alkyl with an attached 3-pyridyl group, C₁₋₁₀ alkyl with an attached 2-pyridyl group, C₁₋₁₀ alkyl with an attached cyclohexyl group, —NH—CH₂CH₂-(4-hydroxyphenyl), —NH—CH₂CH₂-(3-indolyl), C₁₋₁₀ alkyl with an attached 2-furyl group, C₁₋₁₀ alkyl with an attached 3-furyl group, and C₁₋₅ alkyl with an attached phenyl and a hydroxyl attached to the C₁₋₅ alkyl.

In one embodiment, R₁₀ and R₁₁ are selected independently from the group consisting of H, C₁₋₁₀ alkyl, phenyl, nitrophenyl, and C₁₋₁₀ alkyl substituted with phenyl.

In one embodiment, R₃ is selected from the group consisting of M₁, M₂-AA₁, M₂-AA₂-AA₁, and M₂-AA₃-AA₂-AA₁. In one embodiment, R₃ is M₂-AA₁. In one embodiment, R₃ is M₂-AA₂-AA₁. In one embodiment, R₃ is M₂-AA₃-AA₂-AA₁.

In one embodiment, M₂ is Y—O—CO—. In one embodiment, M₂ is Y—O—CO—, wherein Y is benzyl. In one embodiment, M₂ is X—CO—. In one embodiment, M₂ is X—CO—, wherein X is heteroaryl. In one embodiment, M₂ is X—CO—, wherein X is pyrazine.

In one embodiment, R₄ is selected from the group consisting of hydrogen, C₁₋₁₀ alkyl, C₁₋₁₀ alkyl substituted with CONH₂, and C₁₋₁₀ alkyl substituted with CO₂H. In one embodiment, R₄ is hydrogen. In one embodiment, R₄ is C₁₋₁₀ alkyl. In one embodiment, R₄ is methyl. In one embodiment, R₄ is isobutyl. In one embodiment, R₄ is C₁₋₁₀ alkyl substituted with CONH₂. In one embodiment, R₄ is isobutyl. In one embodiment, R₄ is CH₂CONH₂. In one embodiment, R₄ is C₁₋₁₀ alkyl substituted with CO₂H. In one embodiment, R₄ is CH₂CO₂H.

In one embodiment, R₅ is selected independently from the group consisting of hydrogen, C₁₋₁₀ alkyl, and C₁₋₁₀ alkyl with a phenyl group attached to the C₁₋₁₀ alkyl. In one embodiment, R₅ is hydrogen. In one embodiment, R₅ is C₁₋₁₀ alkyl. In one embodiment, R₅ is C₁₋₁₀ alkyl with a phenyl group attached to the C₁₋₁₀ alkyl. In one embodiment, R₅ is methyl. In one embodiment, R₅ is benzyl.

The following exemplary compounds are within the scope of the present disclosure:
Cbz-Leu-Leu-ALeu-COH;
Cbz-Leu-Leu-ALeu-COMe;
Cbz-Leu-Leu-ALeu-COBn;
Cbz-Leu-Phe-ALeu-COH;
Cbz-Leu-Phe-ALeu-COMe;
Cbz-Leu-Phe-ALeu-COBn;
Cbz-Leu-Leu-AGly-COH;
Cbz-Leu-Leu-AGly-COMe;
Cbz-Leu-Leu-AAla-COH; and
Cbz-Leu-Leu-AAla-COMe.

The following exemplary compounds are also within the scope of the present disclosure:
Cbz-Asp-Glu-Val-AAsp-COMe; and
Cbz-Asp-Glu-Leu-AAsp-COBn.

The following exemplary compounds are additionally within the scope of the present disclosure:
Cbz-Ala-Ala-AAsn-COH;
Cbz-Ala-Ala-AAsn-COMe; and
Cbz-Ala-Ala-AAsn-COBn.

In one embodiment, the compound of Formula I is Cbz-Leu-Leu-ALeu-COH. In one embodiment, the compound of Formula I is Cbz-Leu-Leu-ALeu-COMe. In one embodiment, the compound of Formula I is Cbz-Leu-Leu-ALeu-COBn. In one embodiment, the compound of Formula I is Cbz-Leu-Phe-ALeu-COH. In one embodiment, the compound of Formula I is Cbz-Leu-Phe-ALeu-COMe. In one embodiment, the compound of Formula I is Cbz-Leu-Phe-ALeu-COBn. In one embodiment, the compound of Formula I is Cbz-Leu-Leu-AGly-COH. In one embodiment, the compound of Formula I is Cbz-Leu-Leu-AGly-COMe. In one embodiment, the compound of Formula I is Cbz-Leu-Leu-AAla-COH. In one embodiment, the compound of Formula I is Cbz-Leu-Leu-AAla-COMe. In one embodiment, the compound of Formula I is Cbz-Asp-Glu-Val-AAsp-COMe. In one embodiment, the compound of Formula I is Cbz-Asp-Glu-Leu-AAsp-COBn. In one embodiment, the compound of Formula I is Cbz-Ala-Ala-AAsn-COH. In one embodiment, the compound of Formula I is Cbz-Ala-Ala-AAsn-COMe. In one embodiment, the compound of Formula I is Cbz-Ala-Ala-AAsn-COBn.

Compositions

Compositions, as described herein, comprising an active compound and an excipient of some sort may be useful in a variety of applications. For example, pharmaceutical compositions comprising an active compound and an excipient may be useful for the treatment or prevention of a cancer, or for the treatment or prevention of a neurodegenerative disease.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semi-solid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), bucally, or as an oral or nasal spray.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof.

Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, varoius gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacilic acid/acrylamide copolymer, polyalkylene oxides such as polyethylene oxide, polypropylene oxide, poly(ethylene oxide-propylene oxide), and a Pluronic polymer, polyoxyethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly(meth)acrylic acid, and esters amide and hydroxyalkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be an injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The active ingredient may be administered in such amounts, time, and route deemed necessary in order to achieve the desired result. The exact amount of the active ingredient will vary from subject to subject, depending on the species, age, and general condition of the subject, the severity of the infection, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some embodiments, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, enteral, sublingual; by intratracheal instillation, bronchial instillation, and/or inhalation; and/or as an oral spray, nasal spray, and/or aerosol. In general the most appropriate route of administration will depend upon a variety of factors including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Methods of Treatment—Neurodegenerative Disease

In some embodiments, the compounds disclosed herein can be used as inhibitors of proteases. In some embodiments, these protease inhibitors can regulate or prevent cell death and are therefore used in methods for the treatment of disorders associated with excessive death (for example, neurodegenerative diseases).

In one aspect, provided herein is a method of treating a neurodegenerative disease in a subject in need thereof, comprising administering to the subject a therapeutically effective amount of a compound of Formula I (or Formula Ia, Formula Ib, Formula Ic, Formula Id, Formula Ie, or Formula If).

In one embodiment, the neurodegenerative disease is selected from stroke, Alzheimer's disease, Huntington's disease, Parkinson's disease, amyotrophic lateral sclerosis (ALS), multiple sclerosis (MS), and spinal muscular atrophy. In one embodiment, the neurodegenerative disease is Alzheimer's disease. In one embodiment, the neurodegenerative disease is Parkinson's disease.

In one embodiment, provided herein is a method of treating Alzheimer's disease in a subject in need thereof, comprising administering to the subject a therapeutically effective amount of a compound of Formula I (or Formula Ia, Formula Ib, Formula Ic, Formula Id, Formula Ie, or Formula If).

In one embodiment, provided herein is a method of treating Parkinson's disease in a subject in need thereof, comprising administering to the subject a therapeutically effective amount of a compound of Formula I (or Formula Ia, Formula Ib, Formula Ic, Formula Id, Formula Ie, or Formula If).

Excessive neuronal apoptosis leads to a variety of diseases, such as, stroke, Alzheimer's disease, Huntington's disease, Parkinson's disease, amyotrophic lateral sclerosis (ALS), multiple sclerosis (MS), and spinal muscular atrophy. In some embodiments, the neurodegenerative disease is selected from Alzheimer's Disease (or early-onset AD); Senile dementia of the Alzheimer's type (or late onset AD); Parkinson's disease; Pick's Disease; Huntington's disease; multiple system atrophy (dementia combined with ataxia, Parkinson's disease, etc.); progressive supranuclear palsy; diffuse Lewy body disease; corticodentatonigral degeneration; hallervorden-Spatz disease; progressive familial myoclonic epilepsy; striatonigral degeneration; progressive supranuclear palsy; torsion dystonia; spasmodic torticollis and other restricted; dyskinesias; familial tremor; Gilles de la Tourette syndrom; Syndromes of progressive ataxia; Cerebellar cortical degeneration; Olivopontocerebellar atrophy; Friedrich's ataxia and related spinocerebellar degenerations; Shy-Drager syndrome; subacute necrotizing encephalopathy; motor neuron disease without sensory changes; amyotrophic lateral sclerosis; infantile spinal muscular atrophy; juvenile spinal muscular atrophy; other forms of familial spinal muscular atrophy; primary lateral sclerosis; hereditary spastic paraplegia; motor neuron disease with sensory changes; peroneal muscular atrophy; hypertrophic interstitial polyneuropathy; other forms of chronic progressive neuropathy; syndromes of progressive visual loss; or retinitis pigmentosa.

In certain embodiments, said disease resulting from increased cell death includes a traumatic brain injury. In certain embodiments, said traumatic brain injury is stroke.

In some embodiments, the subject is a human patient.

Methods of Treatment—Cancer

In some embodiments, the active compounds disclosed herein can be used as protease inhibitors. In some embodiments, these protease inhibitors can regulate or suppress cell proliferation and/or can induce cell death, and can be used against disorders associated with excessive proliferation, for example, a cancer.

In one aspect, disclosed herein is a method of treating a cancer in a subject in need thereof, comprising administering to the subject a therapeutically effective amount of a compound of Formula I (or Formula Ia, Formula Ib, Formula Ic, Formula Id, Formula Ie, or Formula If).

In some embodiments, the methods described herein are used for the treatment or the prevention of a cancer, for example, melanoma, lung cancer (including lung adenocarcinoma, basal cell carcinoma, squamous cell carcinoma, large cell carcinoma, bronchioloalveolar carcinoma, bronchogenic carcinoma, non-small-cell carcinoma, small cell carcinoma, mesothelioma); breast cancer (including triple negative breast cancer (TNBC), ductal carcinoma, lobular carcinoma, inflammatory breast cancer, clear cell carcinoma, mucinous carcinoma, serosal cavities breast carcinoma); colorectal cancer (colon cancer, rectal cancer, colorectal adenocarcinoma); anal cancer; pancreatic cancer (including pancreatic adenocarcinoma, islet cell carcinoma, neuroendocrine tumors); prostate cancer; prostate adenocarcinoma; ovarian carcinoma (ovarian epithelial carcinoma or surface epithelial-stromal tumor including serous tumor, endometrioid tumor and mucinous cystadenocarcinoma, sex-cord-stromal tumor); liver and bile duct carcinoma (including hepatocellular carcinoma, cholangiocarcinoma, hemangioma); esophageal carcinoma (including esophageal adenocarcinoma and squamous cell carcinoma); oral and oropharyngeal squamous cell carcinoma; salivary gland adenoid cystic carcinoma; bladder cancer; bladder carcinoma; carcinoma of the uterus (including endometrial adenocarcinoma, ocular, uterine papillary serous carcinoma, uterine clear-cell carcinoma, uterine sarcomas, leiomyosarcomas, mixed mullerian tumors); glioma, glioblastoma, medulloblastoma, and other tumors of the brain; kidney cancers (including renal cell carcinoma, clear cell carcinoma, Wilm's tumor); cancer of the head and neck (including squamous cell carcinomas); cancer of the stomach (gastric cancers, stomach adenocarcinoma, gastrointestinal stromal tumor); testicular cancer; germ cell tumor; neuroendocrine tumor; cervical cancer; carcinoids of the gastrointestinal tract, breast, and other organs; signet ring cell carcinoma; mesenchymal tumors including sarcomas, fibrosarcomas, haemangioma, angiomatosis, haemangiopericytoma, pseudoangiomatous stromal hyperplasia, myofibroblastoma, fibromatosis, inflammatory myofibroblastic tumor, lipoma, angiolipoma, granular cell tumor, neurofibroma, schwannoma, angiosarcoma, liposarcoma, rhabdomyosarcoma, osteosarcoma, leiomyoma, leiomysarcoma, skin, including melanoma, cervical, retinoblastoma, head and neck cancer, pancreatic, brain, thyroid, testicular, renal, bladder, soft tissue, adenal gland, urethra, cancers of the penis, myxosarcoma, chondrosarcoma, osteosarcoma, chordoma, malignant fibrous histiocytoma, lymphangiosarcoma, mesothelioma, squamous cell carcinoma; epidermoid carcinoma, malignant skin adnexal tumors, adenocarcinoma, hepatoma, hepatocellular carcinoma, renal cell carcinoma, hypernephroma, cholangiocarcinoma, transitional cell carcinoma, choriocarcinoma, seminoma, embryonal cell carcinoma, glioma anaplastic; glioblastoma multiforme, neuroblastoma, medulloblastoma, malignant meningioma, malignant schwannoma, neurofibrosarcoma, parathyroid carcinoma, medullary carcinoma of thyroid, bronchial carcinoid, pheochromocytoma, Islet cell carcinoma, malignant carcinoid, malignant paraganglioma, melanoma, Merkel cell neoplasm, cystosarcoma phylloide, salivary cancers, thymic carcinomas, and cancers of the vagina among others.

In some embodiments, the subject is a human patient.

Additional Methods

In one aspect, provided herein is a method of inhibiting a protease comprising contacting the protease with a compound of Formula I (or Formula Ia, Formula Ib, Formula Ic, Formula Id, Formula Ie, or Formula If), or a pharmaceutically acceptable salt, derivative, hydrate or solvate thereof. Aza-peptide aldehydes and ketones are a novel class of inhibitors designed to target the threonine protease proteasome and clan CD cysteine proteases caspases, legumain, and MALT1 paracaspase.

In one embodiment, the protease comprises a cysteine protease. In one embodiment, the protease comprises a threonine protease. In one embodiment, the protease is caspase 3. In one embodiment, the protease is legumain. In one embodiment, the protease is MALT1. In one embodiment, the compound comprises a proteasome inhibitor.

In some embodiments, the compounds disclosed herein can act as inhibitors of proteases, and can be used to treat cardiovascular, inflammatory, bacterial, viral (HIV), and parasitic diseases.

### EXAMPLES

The following examples are set forth below to illustrate the compounds, compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Preparation of Aza-Peptide Aldehydes and Ketones as Protease Inhibitors

Proteases are enzymes that catalyze the hydrolysis of peptide bonds in proteins, a process called proteolysis. Uncontrolled, excessive proteolysis can lead to a large variety of disease states including cancer, cardiovascular, inflammatory, neurodegenerative (Alzheimer's and Parkinson's diseases), bacterial, viral (HIV), and parasitic diseases. Excessive proteolysis can be stopped by inhibiting the appropriate proteases. Despite the large number of inhibitors that have been designed for proteases, currently only a few classes of inhibitors are specific for their target protease. In this example, aza-peptide aldehydes and ketones are a new class of inhibitors designed to target the threonine protease proteasome and clan CD cysteine proteases caspases, legumain, and MALT1 paracaspase.

Bioinformatic analysis of the mouse and human genomes revealed that at least 600 proteases have been identified to date, which make up ˜2% of the genomes. Proteases have adapted to a wide range of conditions found in complex organisms (variations in pH, etc.) through evolution and are classified based on statistically significant similarities in sequence and structure by Barrett and coworkers in a database called MEROPS.¹ The classification system divides proteases into clans based on catalytic mechanism and families on the basis of common ancestry. The family names stem from the nucleophilic residue in the enzyme's active site. Accordingly, there are five major families, which include serine, cysteine or threonine proteases (amino-terminal nucleophile hydrolases), or aspartic, metalloproteases.

The hydrolysis of the peptide bond by proteases is energetically highly favorable with a Ke_{q} value of 10⁵ (Scheme 1). Hence, proteases are like irreversible biological switches, and proteolysis is a strictly controlled process.

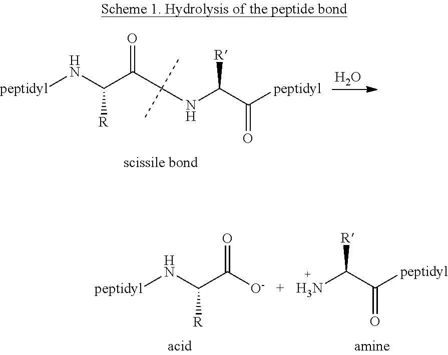

By cleaving proteins, proteases have diverse roles in a large number of key physiological processes such as cell-cycle progression, cell differentiation and growth, blood coagulation and wound healing, immune response and apoptosis (programmed cell death). Uncontrolled, excessive proteolysis can lead to a variety of disease states including cancer, cardiovascular, inflammatory, neurodegenerative (Alzheimer's and Parkinson's diseases), bacterial, viral (HIV), and parasitic diseases. Because excessive proteolysis can be stopped by inhibiting the appropriate proteases, this area is widely explored by pharmaceutical companies. Protease inhibition as a strategy in drug design dates back to the 1950s, where ACE (angiotensin-converting enzyme) inhibitors are the major protease inhibitor success story.² ACE inhibitor drugs have been on the market for more than 20 years with current annual sales exceeding US$6 billion. But in the overall picture of the field of protease inhibition via small molecule drugs, much progress has yet to be made. Many new protease inhibitors are currently in development, with at least 50 different proteases being considered as potential targets.

Besides the use as a therapeutic, one of the biggest areas of research in the protease field is the use of protease inhibitors as biomarkers for diagnostics. This is especially true in cancer diagnostics. For example, the serine protease kallikrein 3, better known as PSA (prostate-specific antigen) has been the major diagnostic marker for prostate cancer for years.³ Likewise, the cysteine protease cathepsin B is a marker for both cancer and arthiritis.⁴ Proteases have also been found to be useful as diagnostic markers for parasitic infections. For example, the cysteine protease cruzain was found to be the major immunogenic protein of Trypanasoma cruzi in Chagas disease.⁵

The drug discovery efforts for protease-targeted therapies will only increase for cancer and neurodegenerative disorders in the near future. A large variety of inhibitors have been described as protease inhibitors,⁶ both as reversible or irreversible inhibitors. A tight-binding, very selective inhibitor is needed. Development of HIV protease inhibitors which meet this criteria provide a standard. Considerable success has also been obtained with irreversible inhibitors to date. Reversible inhibitors of serine and cysteine proteases include peptide aldehydes, trifluoromethyl ketones, boronic acids and α-ketoesters and α-ketoamides. Examples of reversible serine and cysteine inhibitors include:

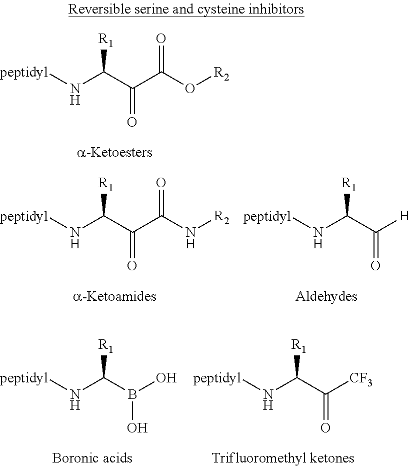

Major classes of irreversible inhibitors include halomethyl ketones, acyloxymethyl ketones, vinyl sulfones, Michael acceptors, phosphonates, epoxysuccinates and epoxy ketones.

Examples of irreversible serine, cysteine, and threonine protease inhibitors include:

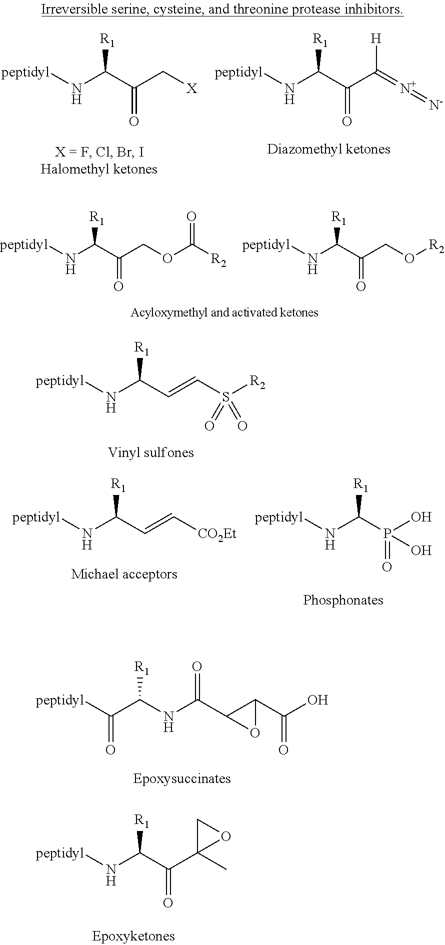

In both reversible and irreversible designs, an electrophilic group, called the warhead, is attached to the ideal peptide substrate of the enzyme to ensure recognition. The most potent inhibitors are those that contain a peptide sequence optimal for the target protease.

Protease Nomenclature

The subsite nomenclature of Schechter& Berger⁷ is used to describe the individual residues of the inhibitors and the corresponding subsites of the protease. The primary substrate binding site of proteases is S1, which recognizes the P1 amino acid residue in substrates and inhibitors. The nomenclature is shown in below:

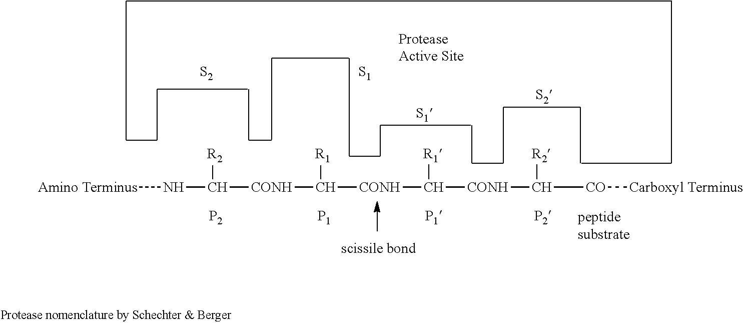

Despite a large number of inhibitors that have been designed for proteases, only a few classes of inhibitors are specific for their target protease. Often times, the embedded electrophilic warhead group is so reactive, regardless of the protease's targeted substrate sequence, the inhibitor reacts with other proteases. One such example is the fluoro- and chloromethyl ketone inhibitors designed for cysteine proteases. The halomethyl ketone functional group is too reactive where it inhibits not only cysteine proteases of different clans, but also serine proteases.

Hence, the challenge is to create a design, where the electrophilic warhead is reactive enough to result in inhibition, and inert enough to be specific for the targeted protease family or clan. Specificity can subsequently be improved by modifying the substrate binding portion of the inhibitor.

Combined efforts in the areas of organic and peptide chemistry, computational methods, and biochemical evaluation have led to novel inhibitors of the proteasome and clan CD cysteine proteases as potential anticancer and antiparasitic agents. The structure of these inhibitors are based on the ideal substrate sequence of the target protease and an α-ketone group as the electrophilic warhead. These inhibitors can inactivate the proteasome and the clan CD cysteine proteases reversibly, while retaining minimal cytotoxicity.

Aza-Peptide Aldehyde and Ketone Design

Aza-peptide ketones are a novel type of transition-state protease inhibitors. These compounds were designed starting with the structure of a target protease substrate structure. The α-carbon of the P1 amino acid residue was replaced with a nitrogen atom to make an aza-amino acid residue and the scissile peptide bond was then replaced with a ketone (when R=alkyl) or an aldehyde (when R═H) moiety:

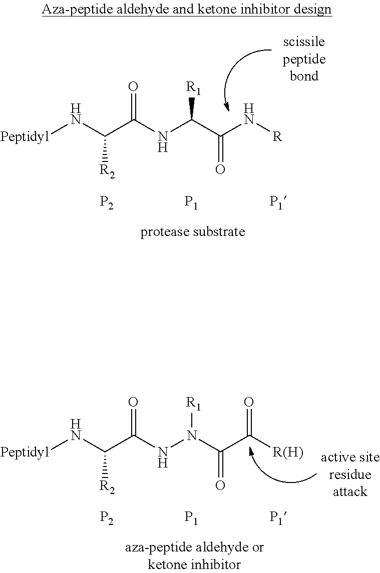

The aza-peptide ketones and aldehydes can inhibit the proteasome and clan CD cysteine proteases reversibly. The active site threonine of the proteasome or the active site cysteine of the caspases, legumain and MALT1 paracaspase attacks the P1′ carbonyl group to form a hemithioacetal intermediate, which is accommodated at by the active site residues, probably through a hydrogen-bonding network (Scheme 2).

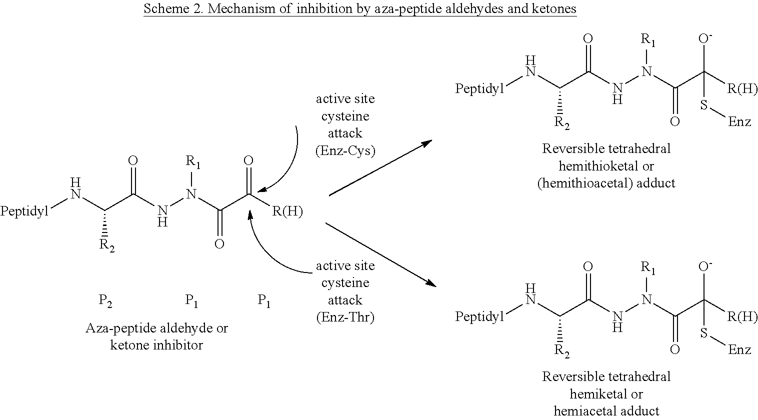

Aza-peptides are first described in the literature as aza-halomethyl ketones.⁸ They inhibited clan CA cysteine proteases much less potently compared to the regular halomethyl ketones. Later, in the mid-2000s, aza-peptide epoxides and aza-peptide Michael acceptors were developed targeting clan CD proteases.⁹

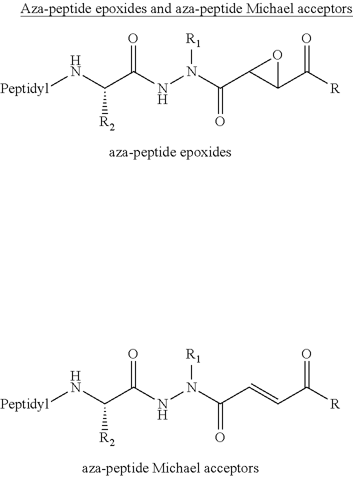

These inhibitors were hugely successful as irreversible inhibitors. Not only did they have second order rate constants in the range of 10⁶ M⁻¹s⁻¹, but they were the first inhibitors differentiating between the two clans of cysteine proteases, where they showed no inhibition against the clan CA proteases papain, cathepsin B and calpains.

Aza-peptides are more rigid than their peptide analogs due to their trigonal planar geometry and the inability of the N2-CO bond to rotate, as opposed to the α-CH—CO bond, which can rotate.

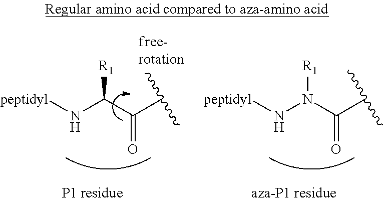

The topology of the active sites of the target proteases plays a crucial role in their ability to bind a substrate. For example, calpains have less flexibility to accommodate the rigid aza-peptide compounds^{9c} whereas caspases tolerate these aza-peptide compounds quite well.^{9a}

A variety of other ketone inhibitors have been reported in the literature as inhibitors of serine, cysteine and threonine proteases. Fluoro-, and chloromethyl ketones, acyloxymethyl ketones, α-ketoaldehydes, α-ketoamides, and epoxyketones are widely investigated as inhibitors for serine proteases, such as chymotrypsin and trypsin; clan CA cysteine proteases, including papain; cathepsins B, H and L; and calpains, the threonine protease proteasome. α-Ketoamides have been especially successful in the inhibition of clan CA member calpains I and II. One α-ketoamide inhibitor AK-295 (Cbz-Leu-D,L-Abu-CONH—(CH₂)3-morpholine) is a potent calpain inhibitor^{9d} that is neuroprotective in models of head trauma^{9e} and focal brains ischemia.^{9f} For animal studies, α-ketoamides have the advantage of exhibiting good membrane permeability.^{9g}

In the compound design and synthesis in this example, an α-keto group (or aldehyde) was incorporated at the aza-P1 residue creating a novel, and “reversed” ketoamide warhead. This new warhead is referred to as an aza-peptide ketone or aza-peptide aldehyde and these inhibitors can be potent and specific reversible inhibitors. Aza-peptide ketone design also allows for extension at the P′ site, where a variety of different groups can be utilized for more selectivity.

The target proteases are the 20S proteasome, and clan CD cysteine proteases caspases, 3, 6 and 8, legumain, and MALT1 paracaspase. Each inhibitor's recognition element can consist of the target protease's ideal substrate sequence to obtain selectivity.

Nomenclature

Aza-peptide ketones (or aldehydes) are abbreviated as peptidyl-ALeu-COR for the proteasome, peptidyl-AAsp-COR for caspases, peptidyl-AAsn-COR for legumain, and peptidyl-AArg for MALT1 paracaspase.

Design and Synthesis of Aza-Peptide Ketones as Reversible Inhibitors Specific for Proteasome

Aza-peptide ketone inhibitors specific for the proteasome were synthesized. This is the first example of an inhibitor design, where an aza-amino acid residue at the P1 position is evaluated with the proteasome. The proteasome active site can tolerate the planarity of the aza-amino acid well, and the aza-peptide ketone inhibitors are potent and selective inhibitors of this enzyme.

The proteasome is a 750 kDa multicatalytic threonine protease that is responsible for the ubiquitin-based degradation of cellular proteins.¹⁰ This multicatalytic complex consists of a 20S proteolytic core particle, which has a cylindrical shape, with the α and β subunits forming four stacked rings, and two 19S regulatory caps which recognize ubiquitinated protein substrates and promote their entry into the central catalytic chamber. In the catalytic chamber of the proteasome, the three major proteolytic activities are chymotrypsin-like, trypsin-like and caspase-like. These catalytic sites cleave after hydrophobic, positively charged, and negatively charged amino acid residues, respectively.

Proteasome inhibition has been validated as a therapeutic approach in the treatment of cancer (multiple myeloma and Non-Hodgkins lymphoma). Thus, proteasome inhibitors are developed that are highly potent and specific and have clinical applicability. In particular, aza-peptide ketone inhibitors are designed targeting the chymotryptic activity of the proteasome. The ideal substrates for the chymotryptic activite site are tripeptides consisting of hydrophobic, non-charged residues such as Z-Leu-Leu-Leu or Z-Leu-Leu-Asn.

Synthesis

The tripeptides with Aza-Leu and Aza-Asn P1 residues were synthesized as previously described.^{9b} The peptidyl methyl ester is reacted with hydrazine to yield a hydrazid derivative. To make the Aza-Leu, the peptide hydrazid is reacted with i-butyraldeyde in ethanol, followed by reduction with sodium cyanoborohydride. Aza-Asn is obtained by reacting the peptide hydrazid with ethyl bromoacetate and NMM in DMF, followed by ammonolysis in methanol and DMF (Scheme 3).

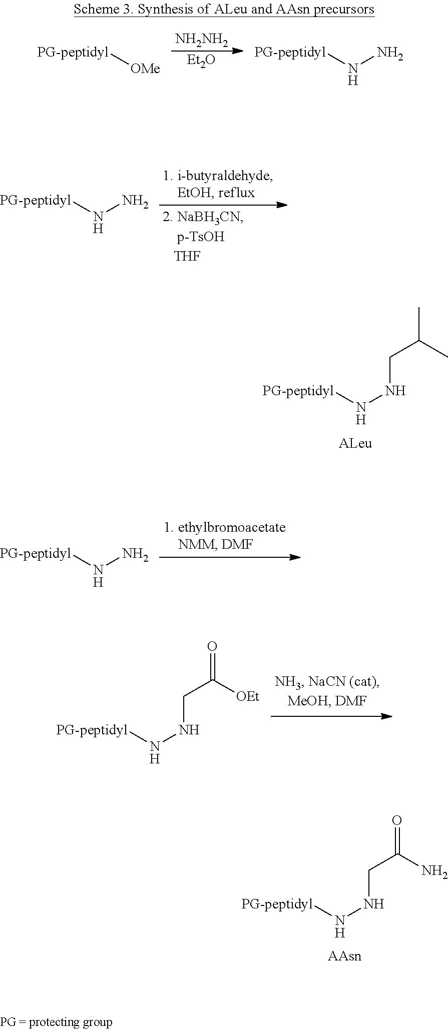

The aza-peptidyl precursors are then coupled to a variety of commercially available ca-ketoacids, and the end product aza-peptide ketones are obtained (Scheme 4).

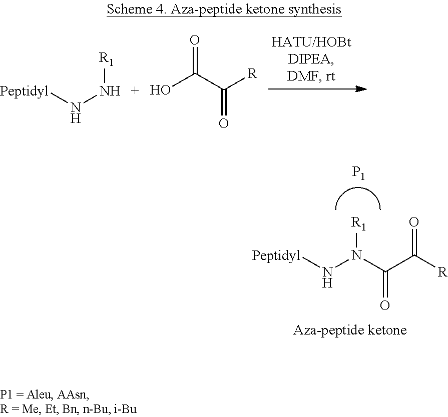

In particular, the following compounds can target the chymotryptic active site of the proteasome (Table 1):

TABLE 1
Aza-peptide ketones targeting the 20S proteasome

| | |
|---|---|
| Cbz-Leu-Leu-ALeu-COH | Cbz-Leu-Leu-AAsn-COH |
| Cbz-Leu-Leu-ALeu-COMe | Cbz-Leu-Leu-AAsn-COMe |
| Cbz-Leu-Leu-ALeu-COEt | Cbz-Leu-Leu-AAsn-COEt |
| Cbz-Leu-Leu-ALeu-COBn | Cbz-Leu-Leu-AAsn-COBn |
| Cbz-Leu-Leu-ALeu-COnBu | Cbz-Leu-Leu-AAsn-COnBu |
| Cbz-Leu-Leu-ALeu-COiBu | Cbz-Leu-Leu-AAsn-COiBu |

Design and synthesis of aza-peptide ketones as reversible inhibitors specific for clan CD cysteine proteases.

Aza-peptide ketone inhibitors are synthesized that are highly specific for clan CD cysteine proteases such as caspases 3, 6, and 8, legumain, and MALT1 paracaspase as potential anticancer and antiparasitic agents. Clan CD cysteine proteases have been shown to tolerate an aza-amino acid residue at the P1 position, which is the primary determinant residue for substrate recognition in this clan.⁹

Caspases, also known as cysteinyl aspartate specific proteases, are a family of 14 members, 11 of which are found in humans. Some caspases are important mediators of inflammation, whereas others are involved in apoptosis (programmed cell death).¹² Excessive neuronal apoptosis leads to a variety of diseases such as stroke, Alzheimer's disease, Huntington's disease, Parkinson's disease, amyotrophic lateral sclerosis (ALS), multiple sclerosis (MS), and spinal muscular atrophy.¹³ Caspases are recognized as novel therapeutic targets for central nervous diseases in which cell death occurs mainly by an apoptosis mechanism.

Legumain, originally identified in leguminous plants¹⁴ and the parasitic blood fluke Schistosoma mansoni 15, has also been discovered in mammals¹⁶ and is associated with bacterial antigen processing and immune disorders.¹⁷

MALT1 (mucosa-associated lymphoid tissue lymphoma translocation 1) paracaspase is a unique protease that plays a vital role in the NF-κB signaling to control lymphocyte survival.¹⁸ The mechanism by which MALT1 promotes NF-κB activation has been intensively studied and shown to involve both its scaffold and proteolytic functions.⁸

The following compounds can target caspases, legumain (Table 2) and MALT1 paracaspase (Table 3):

TABLE 2
Aza-peptide ketones targeting caspases and legumain

| Caspases | Legumain |
|---|---|
| Cbz-Asp-Glu-Val-AAsp-COH | Cbz-Ala-Ala-AAsn-COH |
| Cbz-Asp-Glu-Val-AAsp-COMe | Cbz-Ala-Ala-AAsn-COMe |
| Cbz-Asp-Glu-Val-AAsp-COEt | Cbz-Ala-Ala-AAsn-COEt |
| Cbz-Asp-Glu-Val-AAsp-COBn | Cbz-Ala-Ala-AAsn-COBn |
| Cbz-Asp-Glu-Val-AAsp-COiPr | Cbz-Ala-Ala-AAsn-COiPr |
| Cbz-Asp-Glu-Val-AAsp-CO—i-Bu | Cbz-Ala-Ala-AAsn-CO—i-Bu |

TABLE 3
Aza-peptide ketones targeting MALT1 paracaspase

| | |
|---|---|
| Cbz-Leu-Val-Ser-AArg-COH | Cbz-Leu-Val-Ala-AArg-COH |
| Cbz-Leu-Val-Ser-AArg-COMe | Cbz-Leu-Val-Ala-AArg-COMe |
| Cbz-Leu-Val-Ser-AArg-COEt | Cbz-Leu-Val-Ala-AArg-COEt |
| Cbz-Leu-Val-Ser-AArg-COBn | Cbz-Leu-Val-Ala-AArg-COBn |
| Cbz-Leu-Val-Ser-AArg-COiBu | Cbz-Leu-Val-Ala-AArg-COiBu |
| Cbz-Leu-Val-Ser-AArg-COnBu | Cbz-Leu-Val-Ala-AArg-COnBu |

The corresponding aza-peptide precursors for each target enzyme are synthesized as described in the previous section (Scheme 5), and then they are coupled to the corresponding ca-ketoacids also as previously described (Scheme 4).

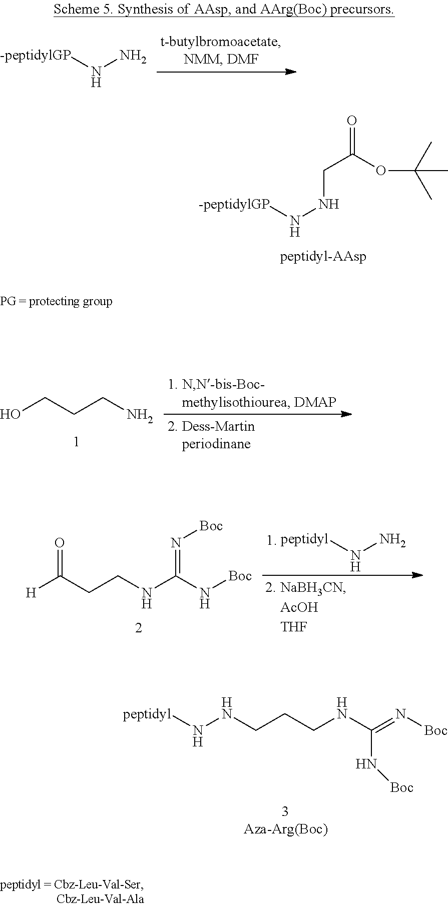

Assaying Inhibitors for Specificity with the Proteasome and Other Proteases.

The inhibitory potency of all the newly synthesized aza-peptide ketone inhibitors is tested against the 20S proteasome and clan CD proteases such as, caspases, legumain, and MALT1 paracaspase. These inhibitors are also tested with clan CA cysteine proteases such as papain, and cathepsin B and the serine proteases elastase and trypsin to check for cross-reactivity. Chromogenic or fluorogenic substrates are used. Proteasome is available commercially. Caspases 3, 6, 7, 8, 9, and 10 are obtained from Professor Guy Salvesen at the Burnham Institute. Legumain and gingipains can be obtained from collaborators in the protease field. Cathepsin B, elastase and trypsin are available commercially.

Synthetic peptide substrates are used to detect enzymes during isolation, to assay enzyme activity and inhibition, and to investigate enzyme specificity. The highest substrate specificity (reflected by low KM values and high k_{cat} rates) is obtained when the peptide sequence of the substrate matches the extended substrate binding site of the protease. The three most commonly used synthetic substrates for proteases are peptide thioesters, peptide p-nitroanilides (pNA), and peptide derivatives of 7-amino-4-methylcoumarin (AMC). The thioester substrates are usually more reactive than the corresponding nitroanilides and aminomethylcoumarins and are useful for detecting new, very unreactive and/or trace enzymes.¹⁹ Peptide amide substrates such as nitroanilides and AMC derivatives are quite specific and suitable for inhibition studies on proteasome and various serine and cysteine proteases. Many peptide pNA and AMC substrates for the 20S proteasome (Suc-LLVY-AMC) and various caspases and other serine and cysteine proteases are commercially available.

The aza-peptide ketones inhibit the target enzymes via the formation of a covalent bond. The kinetic constant Kᵢ (the dissociation constant) is measured and used to evaluate the inhibitory potency of the compounds. The reactions are initiated by adding enzyme to the assay buffer containing substrate and various concentrations of the inhibitor. The initial rates are determined from progress curves at early reaction times. The Kᵢ values are calculated from the rates at various inhibitor concentrations with nonlinear least squares fit to the tight binding equations.²⁰ The inhibition by aza-peptide ketones is tested in the assays for competitive reversible inhibitors, and the Kᵢ values are measured.

Generally, for the 20S proteasome the enzymatic hydrolysis of fluorogenic substrate Suc-LLVY-AMC by the 20S proteasome is monitored continuously for chymotrypsin-like (CT-L) activity at λ_{exc}=360, λₑₘ=460 nm for 45 min at 30° C. under standard assay conditions (Assay buffer: pH 7.5, 20 mM Tris, 0.02% w/v SDS, 1 mM EDTA, 2% v/v DMSO). Caspase activities are measured in 100 mM HEPES, 10% sucrose, 0.1% CHAPS, 10 mM DTT, pH 7.5 and 25° C.²¹ Legumain activity is measured in 0.1 M citrate-phosphate buffer at pH 6.8 containing 4 mM DTT with Cbz-AAN-AMC as the substrate^{9b}. Activity of the MALT1 active fragment (aa325-769) is detected by cleavage of the tetra-peptide substrate LRSR-AMC (7-amino-4-methylcoumarin) that results in fluorescence emission when AMC is released. IC₅₀ values are determined for the new compounds by dose-dependent inhibition. In parallel, well-characterized MALT1 inhibitors (e.g. Z—VRPR-FMIK, mepazine, MI-2) are used as reference compounds. Cathepsin B activity is performed in 0.1 M phosphate, 1.25 mM EDTA, 0.01% Brij, pH 6.0. Papain activity is measured in 10 mM DTT, 50 mM phosphate, pH 6.2. Trypsin activity is performed in 0.046 M Tris/HCl buffer at pH 8.1 with 0.0115 M CaCl₂.

The stability of representative inhibitors is also measured in the assay buffers. This is accomplished simply by incubating the inhibitor in assay buffer for varying periods of time and then measuring the inhibitory potency toward the target protease. If any inhibitor appears to lose its inhibitory potency in buffer, the rate of the hydrolysis or decomposition reactions is then measured.

### REFERENCES CITED IN THIS EXAMPLE

1. Rawlings, N. D.; Waller, M.; Barrett, A. J.; Bateman, A., MEROPS: the database of proteolytic enzymes, their substrates and inhibitors. Nucleic Acids Res 2013.
2. Abbenante, G.; Fairlie, D. P., Protease inhibitors in the clinic. Med Chem 2005, 1 (1), 71-104.
3. Dixon, S. C.; Knopf, K. B.; Figg, W. D., The control of prostate-specific antigen expression and gene regulation by pharmacological agents. Pharmacol Rev 2001, 53 (1), 73-91.
4. Lai, W. F.; Chang, C. H.; Tang, Y.; Bronson, R.; Tung, C. H., Early diagnosis of osteoarthritis using cathepsin B sensitive near-infrared fluorescent probes. Osteoarthritis Cartilage 2004, 12 (3), 239-44.
5. Cazzulo, J. J., Stoka, V. & Turk, V., The major cysteine proteinase of Trypanosoma cruzi: a valid target for chemotherapy of Chagas disease. Curr. Pharm. Des. 2001, 7, 1143-1156.
6. Powers, J. C.; Asgian, J. L.; Ekici, O. D.; James, K. E., Irreversible inhibitors of serine, cysteine, and threonine proteases. Chem Rev 2002, 102 (12), 4639-750.
7. Schechter, I.; Berger, A., On the size of the active site in proteases. I. Papain. Biochem Biophys Res Commun 1967, 27 (2), 157-62.
8. Giordano, C. C. R.; Gallina, C.; Consalvi, V.; Scandurra, R.; Chiaia Noya, F.; Franchini, C. Synthesis andinhibiting activities of 1-peptidyl-2-haloacetyl hydrazines toward cathepsin B and calpains 1993, p. 297.
9. (a) Asgian, J. L.; James, K. E.; Li, Z. Z.; Carter, W.; Barrett, A. J.; Mikolajczyk, J.; Salvesen, G. S.; Powers, J. C., Aza-peptide epoxides: a new class of inhibitors selective for clan CD cysteine proteases. J Med Chem 2002, 45 (23), 4958-60; (b) Ekici, O. D.; Gotz, M. G.; James, K. E.; Li, Z. Z.; Rukamp, B. J.; Asgian, J. L.; Caffrey, C. R.; Hansell, E.; Dvoraik, J.; McKerrow, J. H.; Potempa, J.; Travis, J.; Mikolajczyk, J.; Salvesen, G. S.; Powers, J. C., Aza-peptide Michael acceptors: a new class of inhibitors specific for caspases and other clan CD cysteine proteases. J Med Chem 2004, 47 (8), 1889-92. (c) Giordano, C. C. R.; Gallina, C.; Consalvi, V.; Scandurra, R.; Chiaia Noya, F.; Franchini, C. Synthesis andinhibiting activities of 1-peptidyl-2-haloacetyl hydrazines toward cathepsin B and calpains 1993, p. 297; (d) Li, Z.; Ortega-Vilain, A. C.; Patil, G. S.; Chu, D. L.; Foreman, J. E.; Eveleth, D. D.; Powers, J. C., Novel peptidyl alpha-keto amide inhibitors of calpains and other cysteine proteases. J Med Chem 1996, 39 (20), 4089-98 (e) Saatman, K. E.; Murai, H.; Bartus, R. T.; Smith, D. H.; Hayward, N. J.; Perri, B. R.; McIntosh, T. K., Calpain inhibitor AK295 attenuates motor and cognitive deficits following experimental brain injury in the rat. Proc Natl Acad Sci USA 1996, 93 (8), 3428-33; (f) Bartus, R. T.; Hayward, N. J.; Elliott, P. J.; Sawyer, S. D.; Baker, K. L.; Dean, R. L.; Akiyama, A.; Straub, J. A.; Harbeson, S. L.; Li, Z.; et al., Calpain inhibitor AK295 protects neurons from focal brain ischemia. Effects of postocclusion intra-arterial administration. Stroke 1994, 25 (11), 2265-70. (g) Li, Z.; Patil, G. S.; Golubski, Z. E.; Hori, H.; Tehrani, K.; Foreman, J. E.; Eveleth, D. D.; Bartus, R. T.; Powers, J. C., Peptide alpha-keto ester, alpha-keto amide, and alpha-keto acid inhibitors of calpains and other cysteine proteases. J Med Chem 1993, 36 (22), 3472-80.
10. Coux, O.; Tanaka, K.; Goldberg, A. L., Structure and functions of the 20S and 26S proteasomes. Annu Rev Biochem 1996, 65, 801-47.
11. Nazif, T.; Bogyo, M., Global analysis of proteasomal substrate specificity using positional-scanning libraries of covalent inhibitors. Proc Natl Acad Sci USA 2001, 98 (6), 2967-72.
12. Denault, J. B.; Salvesen, G. S., Caspases: keys in the ignition of cell death. Chem Rev 2002, 102 (12), 4489-500.
13. Yuan, J.; Yankner, B. A., Apoptosis in the nervous system. Nature 2000, 407 (6805), 802-9.
14. Mintz, K.; Shutov, A. D., Legumains and their functions in plants. Trends Plant Sci 2002, 7 (8), 340-4.
15. Sajid, M.; McKerrow, J. H.; Hansell, E.; Mathieu, M. A.; Lucas, K. D.; Hsieh, I.; Greenbaum, D.; Bogyo, M.; Salter, J. P.; Lim, K. C.; Franklin, C.; Kim, J. H.; Caffrey, C. R., Functional expression and characterization of Schistosoma mansoni cathepsin B and its trans-activation by an endogenous asparaginyl endopeptidase. Mol Biochem Parasitol 2003, 131 (1), 65-75.
16. Chen, J. M.; Dando, P. M.; Rawlings, N. D.; Brown, M. A.; Young, N. E.; Stevens, R. A.; Hewitt, E.; Watts, C.; Barrett, A. J., Cloning, isolation, and characterization of mammalian legumain, an asparaginyl endopeptidase. J Biol Chem 1997, 272 (12), 8090-8.
17. Manoury, B.; Hewitt, E. W.; Morrice, N.; Dando, P. M.; Barrett, A. J.; Watts, C., An asparaginyl endopeptidase processes a microbial antigen for class II MHC presentation. Nature 1998, 396 (6712), 695-9.
18. (a) Hailfinger, S., G. Lenz and M. Thome (2014). “Targeting B-cell lymphomas with inhibitors of the MALT1 paracaspase.” Curr Opin Chem Biol 23: 47-55. (b) Perroud, D. Golshayan, O. Gaide and M. Thome (2014). “Maltl protease inactivation efficiently dampens immune responses but causes spontaneous autoimmunity.” EMBO J 33(23): 2765-2781.
19. Powers, J. C.; Kam, C. M., Peptide thioester substrates for serine peptidases and metalloendopeptidases. Methods Enzymol 1995, 248, 3-18.
20. Morrison, J. F.; Walsh, C. T., The behavior and significance of slow-binding enzyme inhibitors. Adv Enzymol Relat Areas Mol Biol 1988, 61, 201-301.
21. Thornberry, N. A.; Peterson, E. P.; Zhao, J. J.; Howard, A. D.; Griffin, P. R.; Chapman, K. T., Inactivation of interleukin-1 beta converting enzyme by peptide (acyloxy)methyl ketones. Biochemistry 1994, 33 (13), 3934-40.

### Example 2. AAsn and AAsp Synthesis

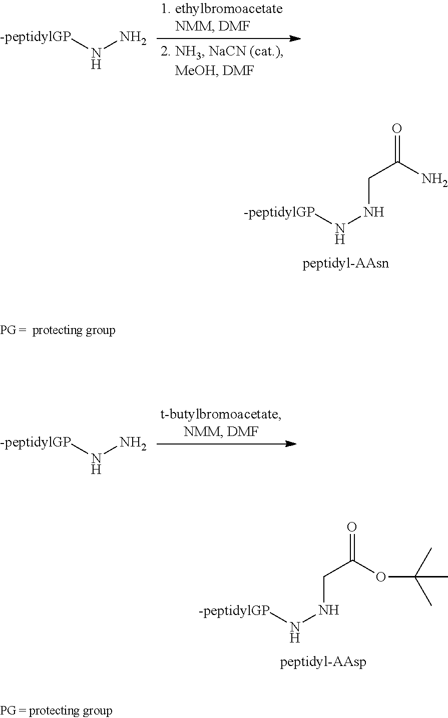

Peptide methyl esters were converted to the corresponding hydrazide by reaction with an excess of hydrazine. The AAsp side chain was introduced by the substitution reaction of the peptide-hydrazid with t-butylbromoacetate.

### Example 3. ALeu and AAsn Synthesis

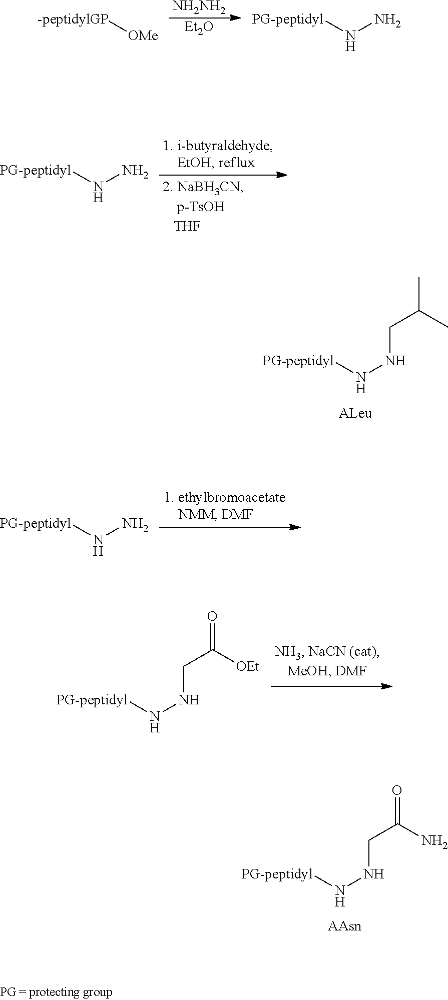

Peptide methyl esters were converted to the corresponding hydrazide by reaction with an excess of hydrazine. To install the leucine aza-amino acid side chain, a reductive amination was carried out on the hydrazide using i-butyraldehyde. For the asparagine aza-amino acid side chain, the hydrazide was reacted with ethylbromoacetate, followed by amination of the ethyl ester using ammonia.

### Example 4. Proposed Aza-Peptide Ketone Inhibition Mechanism

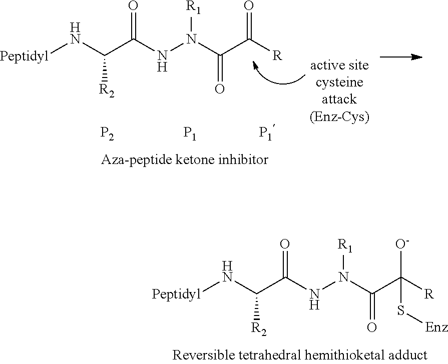

The mechanism of inhibition for cysteine proteases is proposed to occur through the nucleophilic addition of the cysteine thiol to the aldehyde or ketone carbonyl of the aza-peptide ketone warhead. This resulting hemithioacetal or hemithioketal will lead to the reversible inhibition of the cysteine residue. Likewise, the inhibition of the proteasome is proposed to occur through the nucleophilic attack of the threonine oxygen to the carbonyl aldehyde or ketone resulting in a reversible hemiacetal or hemiketal adduct.

### Example 5. Aza-Peptide Aldehyde and Ketone Synthesis

Aza-Peptide Aldeyhde and Ketone Design

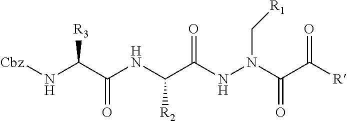

General Peptide Coupling Reaction

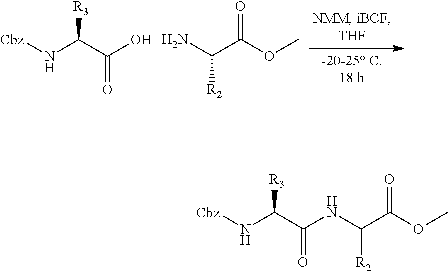

### EXAMPLES

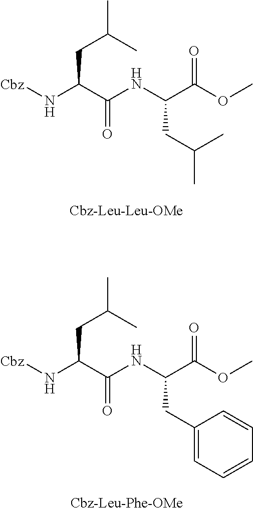 [SMILES: "Cbz-Leu-Leu-OMe" ; "Cbz-Leu-Phe-OMe" ; COC(=O)[C@H](CC(C)C)NC(=O)[C@H](CC(C)C)NC(=O)OCc1ccccc1 ; COC(=O)[C@H](Cc1ccccc1)NC(=O)[C@H](CC(C)C)NC(=O)OCc1ccccc1]

Peptide backbones are synthesized using the i-butylchloroformate method with the appropriate Cbz and methyl ester protected amino acids. The aza-amino acid precursors were synthesized by one of the following methods:

Aza-Gly: The methyl ester of the appropriate peptide backbone was stirred with excess hydrazine for 16 h to give the corresponding Aza-Gly peptide.

Aza-Leu: The methyl ester of the appropriate peptide backbone was stirred with excess hydrazine for 16 h to give the corresponding hydrazide. The hydrazide was subjected to reductive amination using i-butyraldehyde to afford the Aza-Leu peptide.

Aza-Ala: The methyl ester of the appropriate peptide backbone was hydrolyzed using 1 M NaOH. The carboxylic acid was coupled to boc-protected methyl hydrazine using an EDC coupling. The boc group was removed using trifluoroacetic acid to afford the Aza-Ala peptide.

General Warhead Incorporation

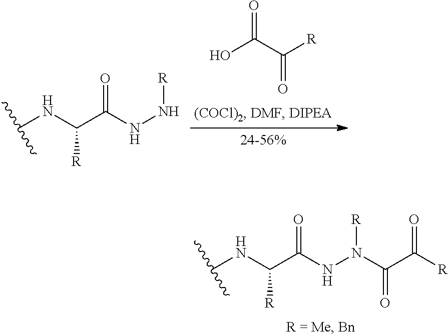

Substituted Hydrazid Synthesis

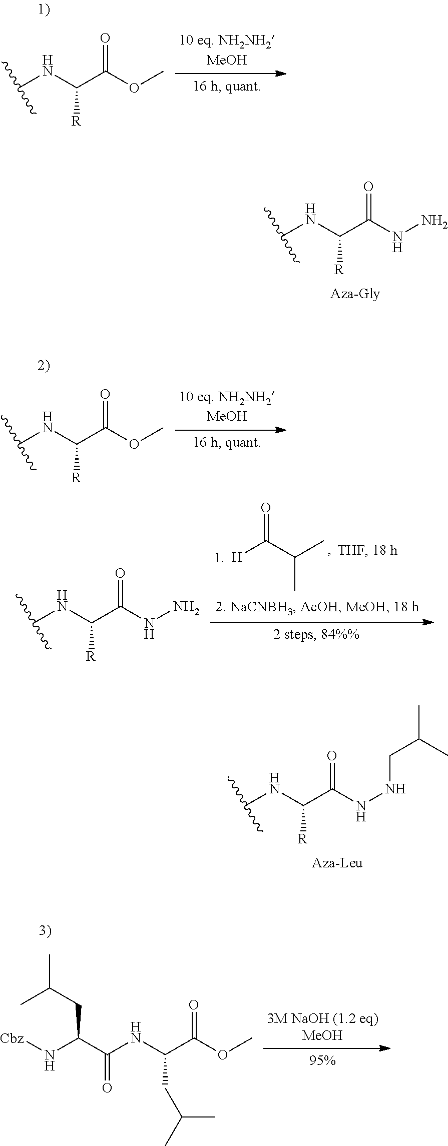

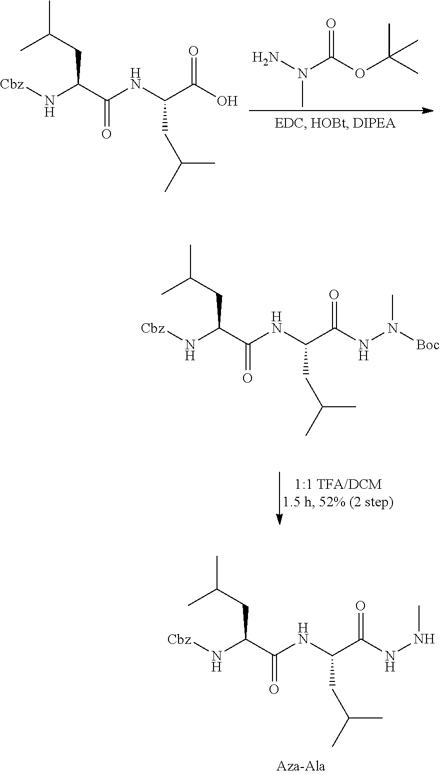

The electrophilic warheads were installed by the following general reactions: The conversion of the appropriate carboxylic acid to the corresponding acid chloride was carried out using oxalyl chloride or thionyl chloride. The acid chlorides were then coupled to the appropriate aza-peptide in the presence of amine base to afford the final inhibitor compounds.

The inhibitors incorporate an electrophilic carbonyl that inhibits the target protease by the proposed nucleophilic addition to the carbonyl carbon. The aza-peptide inhibitor's specificity is designed to arise from the incorporation of amino acid residues found in the target proteases' natural substrates.

Non-limiting examples of aza-peptide aldehyde and ketone compounds include:

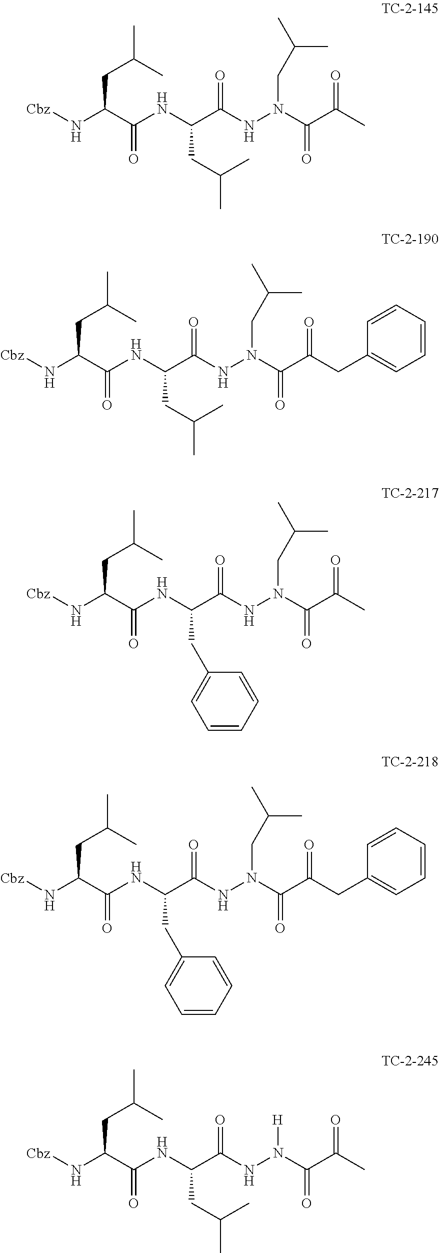 [SMILES: CC(=O)C(=O)N(CC(C)C)NC(=O)[C@H](CC(C)C)NC(=O)[C@H](CC(C)C)NC(=O)OCc1ccccc1 ; CC(C)C[C@H](NC(=O)OCc1ccccc1)C(=O)N[C@@H](CC(C)C)C(=O)NN(CC(C)C)C(=O)C(=O)Cc1ccccc1 ; CC(=O)C(=O)N(CC(C)C)NC(=O)[C@H](Cc1ccccc1)NC(=O)[C@H](CC(C)C)NC(=O)OCc1ccccc1 ; CC(C)C[C@H](NC(=O)OCc1ccccc1)C(=O)N[C@@H](Cc1ccccc1)C(=O)NN(CC(C)C)C(=O)C(=O)Cc1ccccc1 ; CC(=O)C(=O)NNC(=O)[C@H](CC(C)C)NC(=O)[C@H](CC(C)C)NC(=O)OCc1ccccc1]

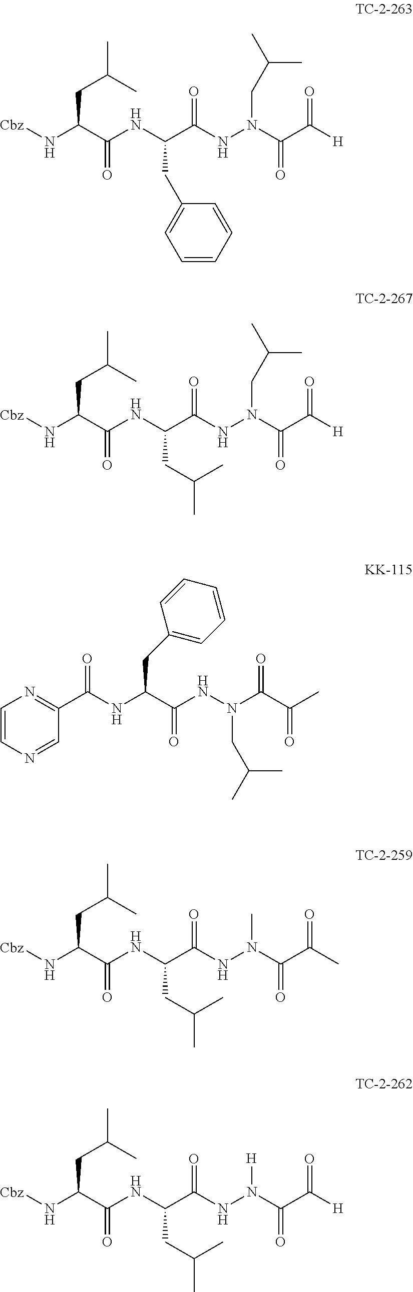 [SMILES: CC(C)C[C@H](NC(=O)OCc1ccccc1)C(=O)N[C@@H](Cc1ccccc1)C(=O)NN(CC(C)C)C(=O)C=O ; CC(C)C[C@H](NC(=O)OCc1ccccc1)C(=O)N[C@@H](CC(C)C)C(=O)NN(CC(C)C)C(=O)C=O ; CC(=O)C(=O)N(CC(C)C)NC(=O)[C@H](Cc1ccccc1)NC(=O)c1cnccn1 ; CC(=O)C(=O)N(C)NC(=O)[C@H](CC(C)C)NC(=O)[C@H](CC(C)C)NC(=O)OCc1ccccc1 ; CC(C)C[C@H](NC(=O)OCc1ccccc1)C(=O)N[C@@H](CC(C)C)C(=O)NNC(=O)C=O]

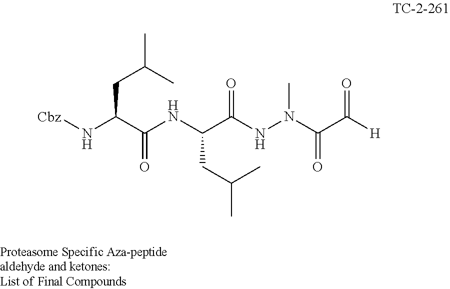 [SMILES: CC(C)C[C@H](NC(=O)OCc1ccccc1)C(=O)N[C@@H](CC(C)C)C(=O)NN(C)C(=O)C=O]

Aza-Peptide Aldeyde Synthesis

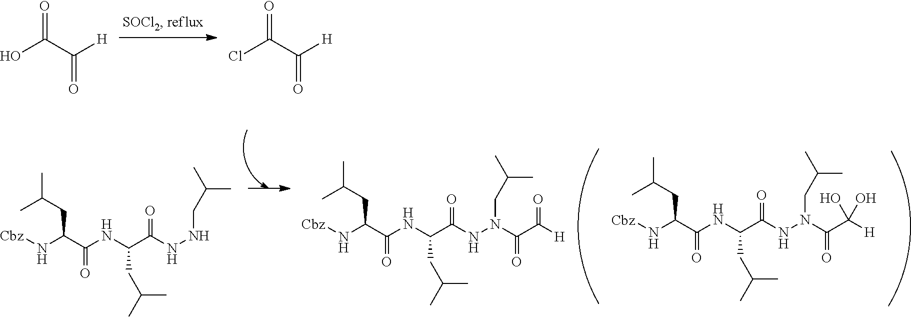

### Example 6. Aza-Peptide Ketones Compared to Bortezomib

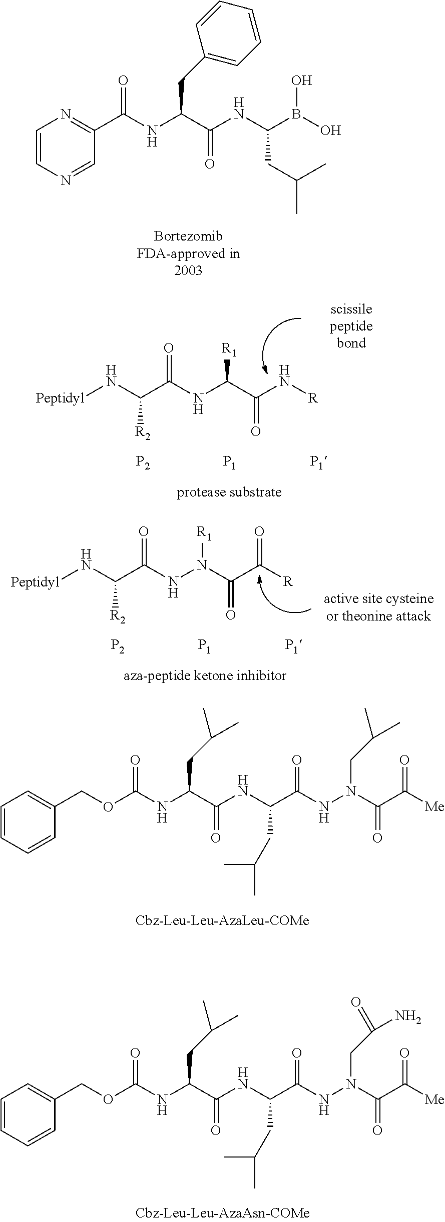

### Example 7. Synthesis of Aza-Peptide Precursors

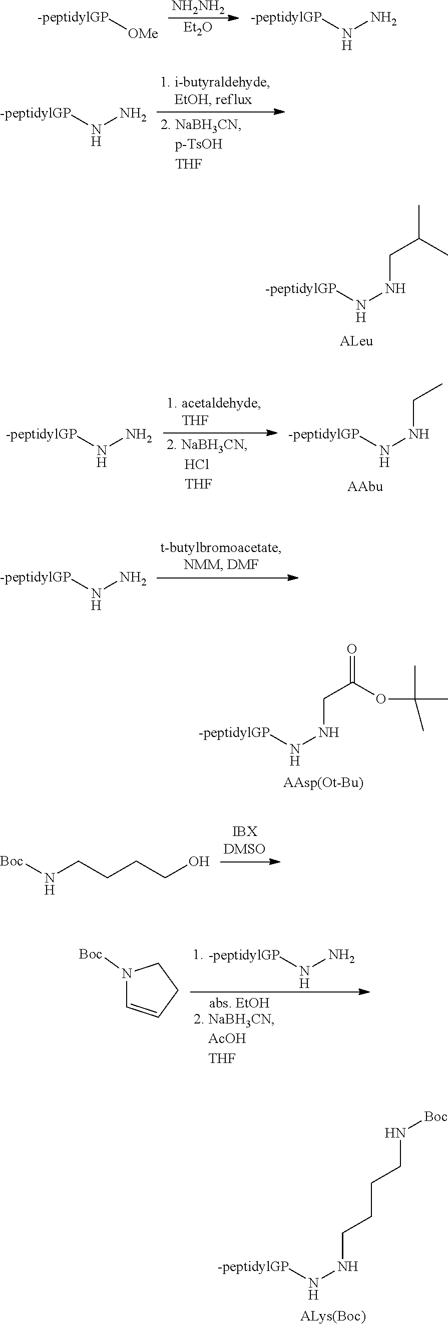

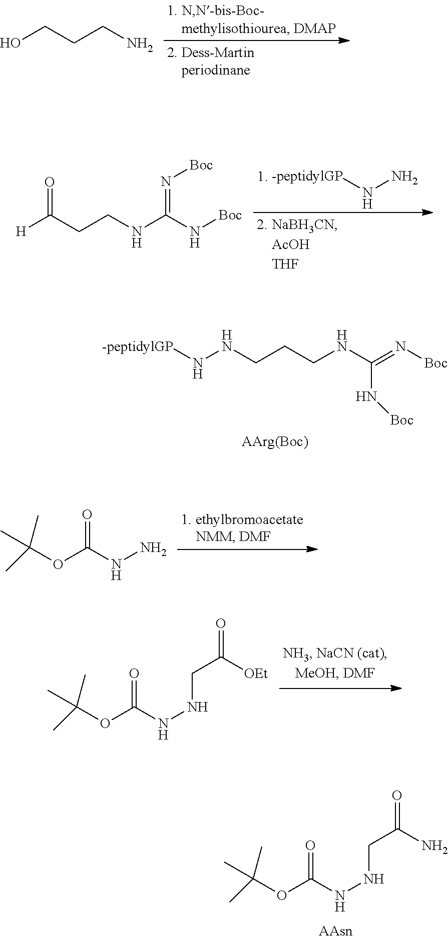

The aza-amino acid precursors are synthesized by one of the following methods:

Aza-Leu: The methyl ester of the appropriate peptide backbone is stirred with excess hydrazine for 16 h to give the corresponding hydrazide. The hydrazide was subjected to reductive amination using i-butyraldehyde to afford the Aza-Leu peptide.

Aza-Abu: The methyl ester of the appropriate peptide backbone is stirred with excess hydrazine for 16 h to give the corresponding hydrazide. The hydrazide is subjected to reductive amination using acetaldehyde to afford the Aza-Abu peptide.

Aza-Asp(Ot-Bu): The methyl ester of the appropriate peptide backbone is stirred with excess hydrazine for 16 h to give the corresponding hydrazide. The hydrazide is reacted with t-butylbromoacetate to afford the Aza-Asp(OtBu) peptide.

Aza-Lys(Boc): Boc-protected amino butanol is oxidized with IBX to give the boc-protected dihydropyrrole. The dihydropyrrole is reacted with the appropriate peptide hydrazid to afford the Aza-Lys(boc) peptide.

Aza-Arg(Boc): Treatment of amino propanol with N,N′-bis-Boc-methylisothiourea followed by oxidation with DMP furnishes the guanidine aldehyde which is used for a reductive amination with the appropriate peptidyl hydrazide to afford the Aza-Arg(Boc) peptide.

Aza-Asn: N-Boc-hydrazine is reacted with ethylbromoacetate, followed by amidation of the ethyl ester using ammonia to afford the Aza-Asn side chain.

### Example 8. Tetrapeptide Aza-Arg Synthesis for MALT1 Inhibitors

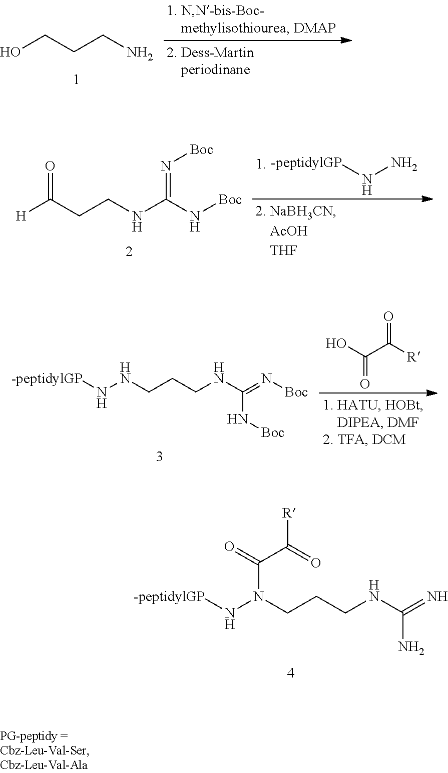

Treatment of amino propanol with N,N′-bis-Boc-methylisothiourea followed by oxidation with DMP furnishes the guanidine aldehyde which is used for a reductive amination with the appropriate peptidyl hydrazide to afford the Aza-Arg(Boc) peptide. Coupling of the warhead to the Aza-peptide is carried out by HATU coupling of the appropriate carboxylic acid to the hydrazide to afford the Aza-Arg peptide inhibitor.

### Example 9. Syntheses of Aza-Peptide Aldehyde and Ketones

Materials and Methods.

Monopeptidyl methyl esters were purchased from Bachem, Torrance, Ca. Di and tripeptides were synthesized using standard coupling procedures such as the mixed anhydride method. The ¹H NMR spectra were obtained using a Bruker Avance III 400 MHz spectrometer. Electrospray ionization (ESI) and high-resolution mass spectrometry were performed using a Bruker MicrOTOF mass spectrometer.

Abbreviations.

The following abbreviations have been used: AMC, 7-amino-4-methyl coumarin; AAla, aza-alanine residue; AAsp, aza-aspartic acid residue; AAsn, aza-asparagine residue; AGly, aza-glycine residue; ALeu, aza-leucine residue; Cbz, Ph-CH₂—OCO—; Pz, pyrazinyl; DCM, dichloromethane; DMF, N,N-dimethylformamide; DMSO, dimethyl sulfoxide; EtOAc, ethyl acetate; iBCF, isobutyl chloroformate; MeOH, methanol; NMM, 4-methylmorpholine; RT, room temperature; THF, tetrahydrofuran.

The Mixed Anhydride Method.

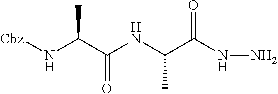 [SMILES: C[C@H](NC(=O)OCc1ccccc1)C(=O)N[C@@H](C)C(=O)NN]

### Benzyl((S)-1-(((S)-1-hydrazinyl-1-oxopropan-2-yl) carbamate (Cbz-Ala-Ala-NHNH₂)

A solution of methyl ((benzyloxy)carbonyl)-L-alanyl-L-alaninate (2 g, 6.4 mmol) in MeOH (50 mL) was treated with the drop wise addition of hydrazine (2.12 mL, 64 mmol). The reaction was stirred at RT for 18 h. MeOH and excess hydrazine were removed in vacuo and remaining white solid was collected by vacuum filtration and washed with EtOAc (1.68 g, 5.4 mmol). Yield: 85% ¹H NMR (DMSO, 400 MHz) δ 1.18 (s, 3H), 1.19 (s, 3H), 4.04-4.11 (m, 1H), 4.20-4.26 (m, 2H), 4.97-5.07 (m, 2H), 7.29-7.40 (m, 5H), 7.40-7.48 (d, 1H), 7.87-7.93 (d, 1H), 9.05 (s, 1H).

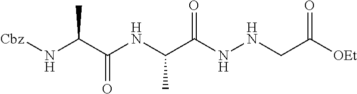 [SMILES: CCOC(=O)CNNC(=O)[C@H](C)NC(=O)[C@H](C)NC(=O)OCc1ccccc1]

### Ethyl((S)-2-((S)-2(((benzyloxy)carbonyl)amino)propanamido)propanamido) glycinate (Cbz-Ala-Ala-NHNHCH₂COOEt)

A solution of benzyl ((S)-1-(((S)-1-hydrazinyl-1-oxopropan-2-yl)amino)-1-oxopropan-2-yl)carbamate (1.68 g, 5.4 mmol) and N-methylmorpholine (0.65 mL, 5.9 mmol) in DMF (8 mL) was chilled to −10° C. and treated with the drop wise addition of ethyl bromoacetate (0.65 mL. 5.9 mmol). The reaction was stirred at −10° C. for 30 min, then warmed to RT and stirred 16 h. The reaction was then concentrated in vacuo, and the resulting residue was purified by silica gel chromatography (1:9 MeOH/DCM) to afford a white solid (375 mg, 0.95 mmol). Yield: 200%; ¹H NMR: (DMSO, 400 MHz) δ: 1.15-1.22 (m, 6H), 1.35-1.44 (m, 3H), 3.48 (d, 2H), 4.01-4.14 (m, 2H), 4.18-4.26 (q, 1H), 5.02 (s, 2H), 5.18 (q, 1H), 7.28-7.41 (m, 5H), 7.41-7.47 (d, 1H), 7.92 (d, 1H), 9.34 (d, 1H).

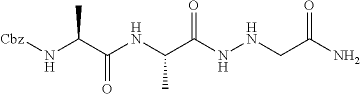 [SMILES: C[C@H](NC(=O)OCc1ccccc1)C(=O)N[C@@H](C)C(=O)NNCC(N)=O]

### Benzyl ((S)-1-(((S)-1-(2-(2-amino-2-oxoethyl)hydrazinyl)-1-oxopropan-2-yl)amino)-1-oxopropan-2-yl)carbamate (Cbz-Ala-Ala-NHNHCH₂COONH₂)

A 7 M solution of ammonia in MeOH (13.5 mL, 94.5 mmol) was added to a 0° C. solution of ethyl ((S)-2-((S)-2-(((benzyloxy)carbonyl)amino)propanamido)propanamido)glycinate (375 mg, 0.95 mmol) in DMF (0.4 mL). NaCN (5 mg, 0.095 mmol) was added and the reaction vessel was sealed. The mixture was stirred at 0° C. for 2 h, then warmed to RT and stirred 48 h. The reaction was concentrated in vacuo and the product was precipitated with 9:1 DCM:MeOH to afford a white solid (343 mg, 0.93 mmol). Yield: 98% ¹H NMR (DMSO, 400 MHz) δ 1.18 (s, 3H), 1.20 (s, 3H), 3.2 (d, 2H). 4.01-4.11 (m, 1H), 4.15-4.24 (m, 1H), 4.97-5.08 (m, 2H), 5.22 (t, 1H), 7.12 (s, 1H), 7.30-7.47 (m, 5H), 7.98 (d, 1H), 9.33 (s, 1H).

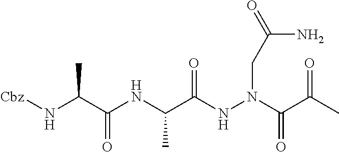 [SMILES: CC(=O)C(=O)N(CC(N)=O)NC(=O)[C@H](C)NC(=O)[C@H](C)NC(=O)OCc1ccccc1]

### Benzyl ((S)-1-(((S)-1-(2-(2-amino-2-oxoethyl)-2-(2-oxopropanoyl)hydrazinyl)-1-oxopropan-2-yl)amino)-1-oxopropan-2-yl)carbamate (Cbz-Ala-Ala-AAsn-COMe)

A 0° C. of pyruvic acid (0.15 mL, 0.62 mmol) in DCM (10 mL) was treated with oxalyl chloride (0.06 mL, 0.66 mmol) followed by one drop of DMF. The reaction was stirred at 0° C. for 30 min, then warmed to RT and stirred an additional 1.5 h. To this mixture was added DIPEA (0.14 mL, 0.82 mmol) followed by a solution of benzyl ((S)-1-(((S)-1-(2-(2-amino-2-oxoethyl)hydrazinyl)-1-oxopropan-2-yl)amino)-1-oxopropan-2-yl)carbamate (150 mg, 0.41 mmol) in 1:2 DCM:DMF (1 mL). The reaction was stirred 16 h, then concentrated in vacuo and purified by silica chromatography (1:9 MeOH/DCM) to afford a white solid (24 mg, 13%). %). ¹H NMR (DMSO, 400 MHz) δ: 10.41 (s, 1H), 9.05 (s, 1H), 8.11-8.00 (m, 1H), 7.55-7.26 (m, 6H), 5.03 (s, 2H), 4.13-4.05 (m, 2H), 3.18 (d, J=5.24 Hz, 2H), 2.03-1.98 (m, 3H), 1.55-1.42 (m, 6H); MS: Found [C₁₉H₂₅N₅O₇+Na]⁺ 458.1634.

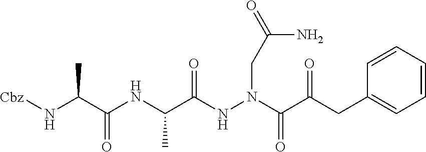 [SMILES: C[C@H](NC(=O)OCc1ccccc1)C(=O)N[C@@H](C)C(=O)NN(CC(N)=O)C(=O)C(=O)Cc1ccccc1]

### Benzyl((S)-1-(((S)-1-(2-(2-amino-2-oxoethyl)-2-(2-oxo-3-phenylpropanoyl) hydrazinyl)-1-oxopropan-2-yl)amino)-1-oxopropan-2-yl)carbamate (Cbz-Ala-Ala-AAsn-COBn)

A solution of phenylpyruvic acid (172 mg, 1.05 mmol) in DCM (3 mL) was treated with oxalyl chloride (0.09 mL, 1.05 mL) followed by DMF (1 drop) the reaction was stirred for 1.5 h at RT, then concentrated in vacuo. The resulting residue was taken up in fresh DCM (5 mL). To this solution was added a solution of benzyl ((S)-1-(((S)-1-(2-(2-amino-2-oxoethyl)hydrazinyl)-1-oxopropan-2-yl)amino)-1-oxopropan-2-yl)carbamate (80 mg, 0.21 mmol) in DCM (1/5 mL) followed by DIPEA (0.07 mL, 0.21 mmol). The reaction was stirred at RT for 16 h, then concentrated in vacuo and purified by silica gel chromatography (2-10% MeOH/DCM) then recrystallized from EtOAc/Et₂O to afford the pure product as a white solid (19 mg, 0.037 mmol, 18%). ¹H NMR (DMSO, 400 MHz) δ: 1.05-1.30 (m, 6H), 4.00 (s, 2H), 4.05-4.13 (m, 1H), 4.17-4.31 (m, 1H), 4.94-5.08 (m, 2H), 7.17-4.40 (m, 12H, ArH and NH₂), 8.13 (d, 1H). ESI MS Found [C₂₅H₂₉N₅O₇Na]⁺: 534.1975 (M+23).

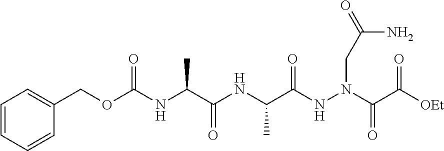 [SMILES: CCOC(=O)C(=O)N(CC(N)=O)NC(=O)[C@H](C)NC(=O)[C@H](C)NC(=O)OCc1ccccc1]

### Ethyl 2-(1-(2-amino-2-oxoethyl)-2-(((benzyloxy)carbonyl)-L-alanyl-L-alanyl)hydrazinyl)-2-oxoacetate: (Cbz-Ala-Ala-AAsn-COOEt)

a solution of benzyl ((S)-1-(((S)-1-(2-(2-amino-2-oxoethyl)hydrazinyl)-1-oxopropan-2-yl)amino)-1-oxopropan-2-yl)carbamate (100 mg, 0.27 mmol) in DCM (4 mL) was chilled to 0° C. To this solution was added DIPEA (0.09 mL, 0.54 mmol) followed by the dropwise addition of ethyl chlorooxoacetate (0.03 mL, 0.27 mmol). The reaction was stirred at 0° C. for 30 min, the warmed to RT and stirred an additional 16 h. The solvent was removed under reduced pressure and the crude product was purified by silica gel chromatography (5% MeOH-DCM) to afford the title product as a white solid (9 mg, 7% yield). ¹H NMR (DMSO, 400 MHz) δ: 10.8 (s, 1H), 8.08 (s, 1H), 7.60-7.18 (m, 7H Ar—H and NH₂), 5.13-4.91 (m, 2H), 4.39-3.92 (m, H), 1.29-1.20 (m, 9H) MS: calc. for: [C₂₀H₂₇N₅O₈+Na]488.1757, Found: [C₂₀H₂₇N₅O₈+Na]⁺488.1749.

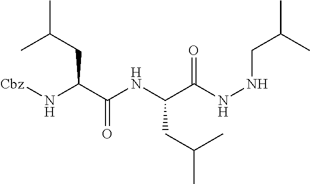 [SMILES: CC(C)CNNC(=O)[C@H](CC(C)C)NC(=O)[C@H](CC(C)C)NC(=O)OCc1ccccc1]

### Benzyl ((S)-1-(((S)-1-(2-isobutylhydrazinyl)-4-methyl-1-oxopentan-2-yl)amino)-4-methyl-1-oxopentan-2-yl)carbamate (Cbz-Leu-Leu-NHNHCH₂CH(CH₃)₂)

A solution of benzyl ((S)-4-methyl-1-(((S)-4-methyl-1-(2-((E)-2-methylpropylidene)hydrazinyl)-1-oxopentan-2-yl)amino)-1-oxopentan-2-yl)carbamate (1 g, 2.24 mmol) in MeOH (25 mL) was treated with acetic acid (0.2 mL) followed by sodium cyanoborohydride (170 mg, 2.69 mmol). The reaction was stirred at 23° C. for 16 h, then the pH was adjusted to ˜10 by addition of 1 M NaOH. Solvent was removed in vacuo, and the residue was dissolved in DCM. The solution was washed with water, brine, dried over Na₂SO₄ and then concentrated in vacuo to afford the product as a white solid (874 mg, 1.95 mmol), 87% yield. ¹H NMR (DMSO, 400 MHz) δ: 0.8-1.05 (m, 18H), 1.42-1.82 (m, 9H), 2.60 (d, 1H), 4.10-4.19 (m, 1H), 4.33-4.42 (q, 1H), 5.10 (s, 2H), 5.30 (d, 1H), 6.47 (d, 1H), 7.28-7.39 (m, 5H), 7.87 (d, 1H). ESI MS: Found [C₂₄H₄₀NO₄+Na]⁺471.2950.

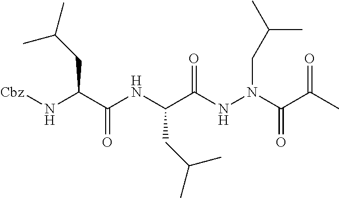 [SMILES: CC(=O)C(=O)N(CC(C)C)NC(=O)[C@H](CC(C)C)NC(=O)[C@H](CC(C)C)NC(=O)OCc1ccccc1]

### Benzyl((S)-1-(((S)-1-(2-isobutyl-2-(2-oxopropanoyl)hydrazinyl)-4-methyl-1-oxopentan-2-yl)amino)-4-methyl-1-oxopentan-2-yl)carbamate (Cbz-Leu-Leu-ALeu-COMe)

A 0° C. solution of pyruvic acid (0.17 mL, 0.69 mmol) in DCM (10 mL) was treated with oxalyl chloride (0.06 mL, 0.72 mmol) followed by DMF (1 drop). The reaction mixture was warmed to RT and stirred 2 h. The solvent was removed in vacuo, and residue was dissolved in fresh DCM (5 mL) and chilled to 0° C. A solution of benzyl ((S)-1-(((S)-1-(2-isobutylhydrazinyl)-4-methyl-1-oxopentan-2-yl)amino)-4-methyl-1-oxopentan-2-yl)carbamate (200 mg, 0.45 mmol) was added, followed by the addition of DIPEA (0.15 mL, 0.9 mmol). The reaction was stirred 1 h at 0° C., then warmed to RT and stirred 16 h. The reaction mixture was diluted with H₂O and extracted with EtOAc. Organic extracts were dried over Na₂SO₄ and concentrated in vacuo. Crude product was purified by silica gel chromatography (20-40% EtOAc-Hexanes) to afford the pure product as a white solid (27 mg, 7.5%). ¹H NMR (DMSO, 400 MHz) δ 0.86-0.98 (m, 18H), 1.40-1.75 (m, 6H), 1.87-1.98 (m, 1H) 2.44 (s, 1H), 3.21-3.56 (m, 2H), 4.05-4.22 (m, 1H), 4.45 (br s, 1H), 5.13 (d, 2H), 5.24 (br s, 1H), 6.38 (d, 1H), 7.28-7.47 (m, 5H), 9.12 (s, 1H); ESI MS: found [C₂₇H₄₂N₄O₆+Na]⁺541.3101.

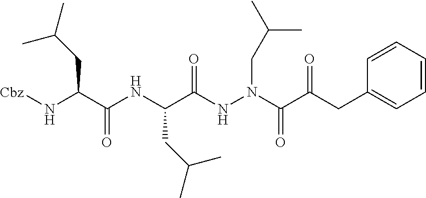 [SMILES: CC(C)C[C@H](NC(=O)OCc1ccccc1)C(=O)N[C@@H](CC(C)C)C(=O)NN(CC(C)C)C(=O)C(=O)Cc1ccccc1]

### Benzyl ((S)-1-(((S)-1-(2-isobutyl-2-(2-oxo-3-phenylpropanoyl)hydrazinyl)-4-methyl-1-oxopentan-2-yl)amino)-4-methyl-1-oxopentan-2-yl)carbamate (Cbz-Leu-Leu-ALeu-COBn)

To a solution of phenylpyruvic acid (73 mg, 0.44 mmol) in DCM (2 mL) was added oxalyl chloride (0.04 mL, 0.44 mmol) followed by DMF (1 drop). The reaction mixture was stirred at RT for 2 h, then concentrated in vacuo. The resulting residue was dissolved in fresh DCM (3 mL) and chilled to 0° C. To this chilled solution was added a solution of benzyl ((S)-1-(((S)-1-(2-isobutylhydrazinyl)-4-methyl-1-oxopentan-2-yl)amino)-4-methyl-1-oxopentan-2-yl)carbamate (168 mg, 0.37 mmol) in DCM (1 mL) followed by DIPEA (0.13 mL, 0.74 mmol). The reaction was warmed to RT and stirred 16 h. The reaction mixture was diluted with H₂O and extracted with DCM. Organic extracts were dried over Na₂SO₄ and concentrated in vacuo. The resulting crude product was purified by silica gel chromatography (5% MeOH/DCM) and recrystallized from EtOAc/Et₂O to afford the pure product as a white solid (38 mg, 17%). ¹H NMR (DMSO, 400 MHz) δ: 0.81-0.95 (m, 18H), 1.22-1.35 (m, 2H) 1.49-1.71 (m, 6H), 1.84-1.99 (m, 1H), 3.20-3.37 (m, 1H), 3.40-3.65 (s, 1H), 3.92-4.20 (m, 3H, CH and Bn), 4.31-4.92 (m, 1H), 5.04-5.14 (m, 2H), 6.06 (d, 1H), 7.15-7.20 (d, 1H), 7.27-7.41 (m, 10H), 8.92 (s, 1H); ESI MS: found [C₃₃H₄₆N₄O₆+Na]⁺617.3308

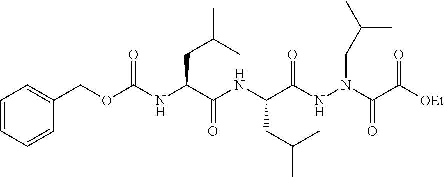 [SMILES: CCOC(=O)C(=O)N(CC(C)C)NC(=O)[C@H](CC(C)C)NC(=O)[C@H](CC(C)C)NC(=O)OCc1ccccc1]

### Ethyl 2-(2-(((benzyloxy)carbonyl)-L-leucyl-L-leucyl)-1-isobutylhydrazinyl)-2-oxoacetate (Cbz-Leu-Leu-ALeu-COOEt)

A solution of benzyl ((S)-1-(((S)-1-(2-isobutylhydrazinyl)-4-methyl-1-oxopentan-2-yl)amino)-4-methyl-1-oxopentan-2-yl)carbamate (100 mg, 0.22 mmol) in DMF (4 mL) was chilled to 0° C. and treated with DIPEA (0.07 mL, 0.44 mmol). To this stirred solution was added chloroethyloxoacetate (0.02 mL, 0.22 mmol) in a drop wise fashion. The reaction was stirred for 30 min, then warmed to RT and stirred an additional 3 h. Water was added and the mixture was extracted with DCM. The organic extracts were washed with brine, dried over Na₂SO₄ and concentrated in vacuo. The crude product was purified by silica gel chromatography (5% MeOH-DCM) to afford the title compound as a white solid (24 mg, 20%). ¹H NMR (DMSO, 400 MHz) δ: 7.41-7.28 (m, 5H), 5.10 (s, 2H), 4.41-4.31 (m, 2H), 4.30-4.17 (m, 2H), 3.86-3.30 (m, 2H), 2.61-2.42 (m, 1H), 1.98-1.71 (m, 4H), 1.54-1.44 (m, 2H), 1.39-1.31 (m, 3H), 0.96-0.89 (m, 18H). ESI MS: cal. for: [C₂₈H₄₄N₄+Na]⁺571.3108, found: [C₃₃H₄₆N₄O₆+Na]⁺571.3116.

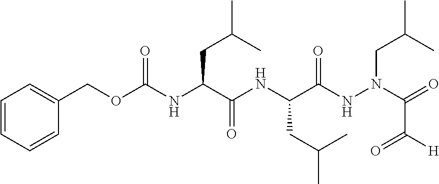 [SMILES: CC(C)C[C@H](NC(=O)OCc1ccccc1)C(=O)N[C@@H](CC(C)C)C(=O)NN(CC(C)C)C(=O)C=O]

### Benzyl ((S)-1-(((S)-1-(2-isobutyl-2-(2-oxoacetyl)hydrazinyl)-4-methyl-1-oxopentan-2-yl)amino)-4-methyl-1-oxopentan-2-yl)carbamate (Cbz-Leu-Leu-ALeu-COH)

A 0° C. solution of benzyl ((S)-1-(((S)-1-(2-isobutylhydrazinyl)-4-methyl-1-oxopentan-2-yl)amino)-4-methyl-1-oxopentan-2-yl)carbamate (50 mg, 0.11 mmol) in dry DCM (2 mL) was treated with the addition of a solution of 2-oxoacetyl chloride (13 mg, 0.14 mmol) in DCM (0.5 mL) followed by DIPEA (0.02 mL, 0.14 mmol). The reaction was warmed to RT and stirred 2 h. H₂O was added, and the mixture was extracted with DCM. The organic layers were washed with sat. NaHCO₃ and concentrated. The product was purified by silica gel chromatography (2% MeOH-DCM) to afford the title product (18 mg, 32% yield). ¹H NMR (DMSO, 400 MHz) δ 9.08 (s, 1H), 7.96 (s, 1H), 7.49-7.24 (m, 5H), 5.01 (s, 2H), 4.37-4.20 (m, 1H), 4.14-4.01 (m, 1H), 3.78-3.66 (m, 1H), 1.77-1.32 (m, 7H), 0.94-0.71 (m, 18H); ESI MS observed [C₂₇H₄₄N₄O₇+Na]⁺ (Methanol hemiacetal adduct): 559.3098.

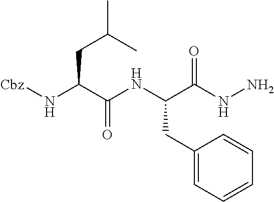 [SMILES: CC(C)C[C@H](NC(=O)OCc1ccccc1)C(=O)N[C@@H](Cc1ccccc1)C(=O)NN]

### Benzyl ((S)-1-(((S)-1-hydrazinyl-1-oxo-3-phenylpropan-2-yl)amino)-4-methyl-1-oxopentan-2-yl)carbamate (Cbz-Leu-Phe-NHNH₂)

A solution of methyl ((benzyloxy)carbonyl)-L-leucyl-L-phenylalaninate (1 g, 2.3 mmol) in methanol (5 mL) was treated with hydrazine (0.76 mL, 23 mmol). The reaction mixture was stirred at RT for 16 h. Excess hydrazine and methanol were removed in vacuo to afford the product as a white solid (998 mg, 2.3 mmol, 99%). ¹H NMR (DMSO, 400 MHz) δ 0.74-0.83 (m, 6H), 1.17-1.37 (m, 2H), 1.41-1.56 (m, 1H), 2.72-2.94 (m, 2H), 4.14-4.27 (m, 1H), 4.19 (s, 2H), 4.40-4.50 (m, 1H), 5.00 (s, 2H), 7.11-7.43 (m, 12H), 7.87 (d, 1H), 9.12 (s, 1H)

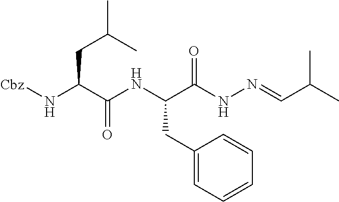 [SMILES: CC(C)/C=N/NC(=O)[C@H](Cc1ccccc1)NC(=O)[C@H](CC(C)C)NC(=O)OCc1ccccc1]

### Benzyl ((S)-4-methyl-1-(((S)-1-(2-((E)-2-methylpropylidene)hydrazinyl)-1-oxo-3-phenylpropan-2-yl)amino)-1-oxopentan-2-yl)carbamate (Cbz-Leu-Phe-NHNCHCH(CH₃)₂)

To a suspension of benzyl ((S)-1-(((S)-1-hydrazinyl-1-oxo-3-phenylpropan-2-yl)amino)-4-methyl-1-oxopentan-2-yl)carbamate (998 mg, 2.3 mmol) in THF (20 mL) was added drop wise isobutyraldehyde (0.23 mL, 2.5 mmol). The reaction mixture was stirred at RT for 16 h, then concentrated in vacuo to afford the title compound as a white solid (1.1 g, 99%). ¹H NMR (DMSO, 400 MHz) shows mix of E and Z isomers. Product was carried on for following transformation without further purification; ESI MS: found [C₂₇H₃₆N₄O₄+Na]⁺503.2613

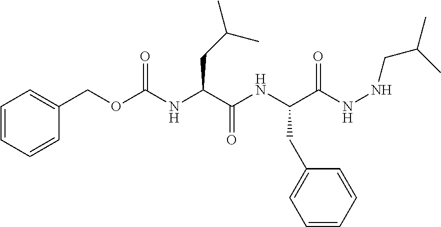 [SMILES: CC(C)CNNC(=O)[C@H](Cc1ccccc1)NC(=O)[C@H](CC(C)C)NC(=O)OCc1ccccc1]

### Benzyl (1-((1-(2-isobutylhydrazinyl)-1-oxo-3-phenylpropan-2-yl)amino)-4-methyl-1-oxopentan-2-yl)carbamate (Cbz-Leu-Phe-NHNHCH₂CH(CH₃)₂)

A solution of benzyl ((S)-4-methyl-1-(((S)-1-(2-((E)-2-methylpropylidene)hydrazinyl)-1-oxo-3-phenylpropan-2-yl)amino)-1-oxopentan-2-yl)carbamate (1.1 g, 2.3 mmol) in MeOH (15 mL) was treated with NaCNBH3 (173 mg, 2.76 mmol) followed by the addition of AcOH (2 drops). The reaction was stirred at RT for 18 h. The pH was adjusted to 10 by the addition of 1 M NaOH. MeOH was removed in vacuo and residue was dissolved in DCM. The mixture was washed with H₂O, brine, dried over Na₂SO₄ and concentrated in vacuo to afford the title compound as a white solid (943 mg, 85%). ¹H NMR (DMSO, 400 MHz) δ: 9.37-9.30 (m, 1H), 8.04-7.91 (m, 1H), 7.43-7.18 (m, 11H), 5.03 (s, 2H), 4.87-4.76 (m, 1H), 4.54-4.40 (m, 1H), 4.07-3.95 (m, 1H), 2.8-2.75 (m, 2H), 2.42-2.28 (m, 2H), 1.60-1.45 (m, 2H), 1.41-1.23 (m, 2H), 0.91-0.77 (m, 12H); ESI MS: found [C₂₇H₃₈N₄O₄+Na]⁺505.2863.

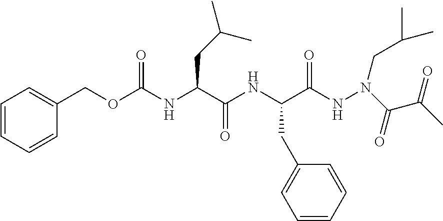 [SMILES: CC(=O)C(=O)N(CC(C)C)NC(=O)[C@H](Cc1ccccc1)NC(=O)[C@H](CC(C)C)NC(=O)OCc1ccccc1]

### Benzyl((S)-1-(((S)-1-(2-isobutyl-2-(2-oxopropanoyl)hydrazinyl)-1-oxo-3-phenylpropan-2-yl)amino)-4-methyl-1-oxopentan-2-yl)carbamate (Cbz-Leu-Phe-ALeu-COMe)

A solution of pyruvic acid (0.09 mL, 1.4 mmol) in dry DCM (2 mL) was treated with oxalyl chloride (0.12 mL, 1.4 mmol) followed by DMF (1 drop, catalytic). The reaction was stirred at RT for 2.5 h, then concentrated under reduced pressure. The resulting residue was dissolved in fresh DCM (5 mL) and chilled to 0° C. A solution of benzyl (1-((1-(2-isobutylhydrazinyl)-1-oxo-3-phenylpropan-2-yl)amino)-4-methyl-1-oxopentan-2-yl)carbamate (200 mg, 0.41 mmol) in DCM (2 mL) was added followed by DIPEA (0.24 mL, 1.4 mmol). The reaction was warmed to RT and stirred 16 h. The mixture was then diluted with water and extracted with DCM. The organic layers were washed with brine, dried over Na₂SO₄ and concentrated. The crude product was purified by silica gel chromatography (2-4% MeOH-DCM) to afford the title product as a white solid (111 mg, 49% yield). ¹H NMR (DMSO, 400 MHz) δ: 10.86 (s, 1H), 8.29 (d, 1H), 7.40-7.13 (m, 11H), 4.99 (s, 2H), 4.58-4.46 (m, 1H), 4.08-3.96 (m, 1H), 3.41-3.24 (m, 2H), 2.92-2.77 (m, 2H), 2.13-1.04 (s, 3H), 1.59-1.47 (m, 1H), 1.38-1.24 (m, 2H), 0.89-0.64 (m, 12H); ESI MS: found [C₃₀H₄₀N₄O₆+Na]⁺575.2814.

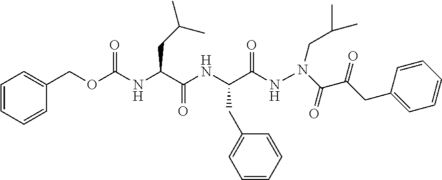 [SMILES: CC(C)C[C@H](NC(=O)OCc1ccccc1)C(=O)N[C@@H](Cc1ccccc1)C(=O)NN(CC(C)C)C(=O)C(=O)Cc1ccccc1]

### Benzyl ((S)-1-(((S)-1-(2-isobutyl-2-(2-oxo-3-phenylpropanoyl)hydrazinyl)-1-oxo-3-phenylpropan-2-yl)amino)-4-methyl-1-oxopentan-2-yl)carbamate (Cbz-Leu-Phe-ALeu-COBn)

A solution of phenylpyruvic acid (230 mg, 1.4 mmol) in dry DCM (2 mL) was treated with oxalyl chloride (0.12 mL, 1.4 mmol) followed by DMF (1 drop, catalytic). The reaction mixture was stirred at RT for 2.5 h, then concentrated under reduced pressure. The resulting residue was dissolved in fresh DCM (5 mL) and chilled to 0° C. A solution of benzyl (1-((1-(2-isobutylhydrazinyl)-1-oxo-3-phenylpropan-2-yl)amino)-4-methyl-1-oxopentan-2-yl)carbamate (200 mg, 0.41 mmol) in DCM (1.5 mL) was added followed by DIPEA (0.24 mL, 1.4 mmol). The reaction was warmed to RT and stirred 16 h. The mixture was diluted with water and extracted with DCM. The organic layers were washed with brine, dried over Na₂SO₄ and concentrated in vacuo. The crude product was purified by silica gel chromatography (2-4% MeOH-DCM) to afford the title product as a white solid (88 mg, 34% yield). ¹H NMR (DMSO, 400 MHz) δ: 10.96 (s, 1H), 8.34 (s, 1H), 7.50-7.15 (m, 16H), 5.00 (s, 2H), 4.70-4.47 (m, 1H), 4.22-3.84 (m, 3H), 3.06-2.61 (m, 4H), 1.63-1.46 (m, 1H), 1.41-1.23 (m, 3H), 0.89-0.73 (m, 12H); ESI MS: found [C₃₆H₄₄N₄O₆+Na]⁺651.3143.

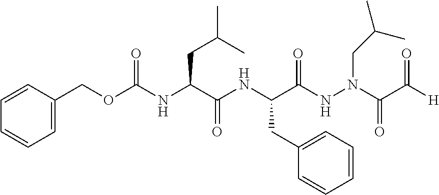 [SMILES: CC(C)C[C@H](NC(=O)OCc1ccccc1)C(=O)N[C@@H](Cc1ccccc1)C(=O)NN(CC(C)C)C(=O)C=O]

### Benzyl ((S)-1-(((S)-1-(2-isobutyl-2-(2-oxoacetyl)hydrazinyl)-1-oxo-3-phenylpropan-2-yl)amino)-4-methyl-1-oxopentan-2-yl)carbamate (Cbz-Leu-Phe-ALeu-COH)

A 0° C. solution of benzyl (1-((1-(2-isobutylhydrazinyl)-1-oxo-3-phenylpropan-2-yl)amino)-4-methyl-1-oxopentan-2-yl)carbamate (50 mg, 0.10 mmol) in dry DCM (3 mL) was treated with a solution of 2-oxoacetyl chloride (12 mg, 0.13 mmol) in DCM (0.5 mL) followed by DIPEA (0.02 mL, 0.13 mmol). The reaction was warmed to RT and stirred 2.5 h. The reaction was diluted with water and extracted with DCM. The organic layers were washed with brine, dried over Na₂SO₄ and concentrated in vacuo. The crude product was recrystallized from EtOAc-Hexanes to afford the title product as a white powder (14 mg, 26% yield). ¹H NMR (DMSO, 400 MHz) δ: 9.37 (s, 1H), 8.07 (br s, 1H), 7.60-6.97 (m, 11H), 5.02 (s, 2H), 4.71-4.27 (m, 1H), 4.19-3.88 (m, 1H), 3.81-3.48 (m, 1H), 3.19-2.73 (m, 3H), 1.65-1.45 (m, 1H), 1.45-1.15 (m, 4H), 0.93-0.57 (m, 12H); ESI MS: found [C₃₀H₄₂N₄O₇+Na]⁺593.2937 (methanol hemiacetal).

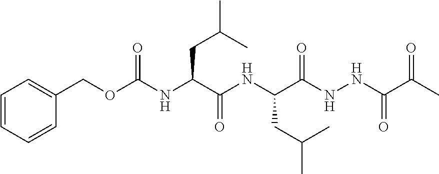 [SMILES: CC(=O)C(=O)NNC(=O)[C@H](CC(C)C)NC(=O)[C@H](CC(C)C)NC(=O)OCc1ccccc1]

### Benzyl((S)-4-methyl-1-(((S)-4-methyl-1-oxo-1-(2-(2-oxopropanoyl)hydrazinyl) pentan-2-yl)amino)-1-oxopentan-2-yl)carbamate (Cbz-Leu-Leu-AGly-COMe)

A solution of pyruvic acid (0.9 mL, 1.34 mmol) in DCM (10 mL) was treated with oxalyl chloride (0.11 mL, 1.34 mmol) followed by DMF (1 drop, catalytic). The reaction was stirred at RT for 3 h, then concentrated in vacuo. The resulting residue was dissolved in fresh DCM (10 mL) and chilled to 0° C. benzyl ((S)-1-(((S)-1-hydrazinyl-4-methyl-1-oxopentan-2-yl)amino)-4-methyl-1-oxopentan-2-yl)carbamate (150 mg, 0.38 mmol) was added followed by DIPEA (0.23 mL, 1.34 mmol). The reaction was stirred at RT for 16 h, then diluted with water and extracted with DCM. The organic extracts were washed with brine, dried over Na₂SO₄ and concentrated in vacuo. The crude residue was purified by silica gel chromatography (5% MeOH-DCM) to afford the title product (12 mg, 7%) as a mixture of ketone and hydrate (seen by ¹H NMR/ESI MS). 1H NMR (DMSO, 400 MHz) δ: 7.54-7.19 (m, 6H), 5.03 (s, 2H), 4.65-4.40 (m, 1H), 4.20-3.99 (m, 1H), 3.84-3.85 (m, 1H), 2.40-1.96 (m, 3H), 1.73-1.57 (m, 2H), 1.56-1.36 (m, 4H), 0.98-0.77 (m, 12H); ESI MS: found [C₂₄H₃₈N₄O₇+Na]⁺517.2439 (methanol hemiacetal).

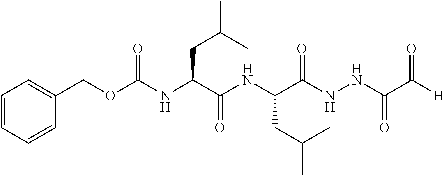 [SMILES: CC(C)C[C@H](NC(=O)OCc1ccccc1)C(=O)N[C@@H](CC(C)C)C(=O)NNC(=O)C=O]

### Benzyl ((S)-4-methyl-1-(((S)-4-methyl-1-oxo-1-(2-(2-oxoacetyl)hydrazinyl)pentan-2-yl)amino)-1-oxopentan-2-yl)carbamate (Cbz-Leu-Leu-AGly-COH)

A 0° C. solution of benzyl ((S)-1-(((S)-1-hydrazinyl-4-methyl-1-oxopentan-2-yl)amino)-4-methyl-1-oxopentan-2-yl)carbamate (65 mg, 0.17 mmol) in dry DCM (3 mL) was treated with a solution of 2-oxoacetyl chloride (18.5 mg, 0.2 mmol) in DCM (0.5 mL) followed by DIPEA (0.03 mL, 0.2 mmol). The reaction was warmed to RT and stirred 4 h, then diluted with sat. NaHCO₃ and extracted with DCM. The organic extracts were washed with water, brine, then dried over Na₂SO₄ and concentrated. The crude product was passed through a plug of silica gel (5% MeOH-DCM) and then recrystallized from EtOAc-Hexanes to afford the title product as a white solid (14 mg, 16% yield). ¹H NMR (DMSO, 400 MHz) δ: 7.39-7.25 (m, 6H), 5.02 (s, 2H), 4.68-4.31 (m, 1H), 4.15-4.01 (m, 1H), 1.74-1.52 (m, 2H), 1.55-1.32 (m, 4H), 0.91-0.80 (m, 12H); ESI MS: found [C₂₃H₃₆N₄O₇+Na]⁺503.2497 (methanol hemiacetal).

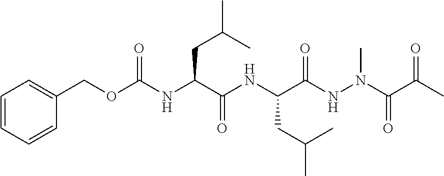 [SMILES: CC(=O)C(=O)N(C)NC(=O)[C@H](CC(C)C)NC(=O)[C@H](CC(C)C)NC(=O)OCc1ccccc1]

### Benzyl ((S)-4-methyl-1-(((S)-4-methyl-1-(2-methyl-2-(2-oxopropanoyl)hydrazinyl)-1-oxopentan-2-yl)amino)-1-oxopentan-2-yl)carbamate (Cbz-Leu-Leu-AAla-COMe)

A solution of pyruvic acid (0.05 mL, 0.73 mmol) in DCM (2 mL) was treated with oxalyl chloride (0.06 mL, 0.73 mmol) followed by DMF (1 drop, catalytic). The reaction was stirred for 45 min then concentrated in vacuo. The resulting residue was dissolved in fresh DCM (5 mL) and chilled to 0° C. A solution of benzyl ((S)-4-methyl-1-(((S)-4-methyl-1-(2-methylhydrazinyl)-1-oxopentan-2-yl)amino)-1-oxopentan-2-yl)carbamate (100 mg, 0.24 mmol) in DCM (1 mL) was added followed by DIPEA (0.13 mmol, 0.73 mmol). The reaction was warmed to RT and stirred 16 h, then diluted with water and extracted with DCM. The organic layer was washed with sat.

NaHCO₃, brine, dried over Na₂SO₄ and concentrated in vacuo. The crude residue was passed through a plug of silica gel (5% MeOH-DCM) and recrystallized from EtOAc/Hexanes to afford the title product as a white solid. ¹H NMR (DMSO, 400 MHz) δ: 10.92-10.67 (m, 1H), 8.39-8.05 (m, 1H), 7.47-7.25 (m, 6H), 5.02 (s, 2H), 4.27-4.15 (m, 1H), 4.14-4.00 (m, 1H), 3.01-2.93 (m, 3H), 2.21-2.12 (m, 3H), 1.67-1.54 (m, 2H), 1.51-1.35 (m, 4H), 0.90-0.81 (m, 12H); ESI MS: found [C₂₄H₃₆N₄O₆+Na]⁺499.2527.

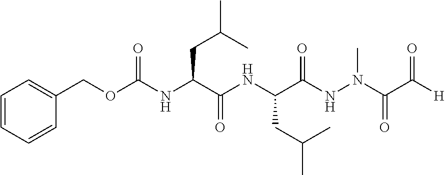 [SMILES: CC(C)C[C@H](NC(=O)OCc1ccccc1)C(=O)N[C@@H](CC(C)C)C(=O)NN(C)C(=O)C=O]

### Benzyl ((S)-4-methyl-1-(((S)-4-methyl-1-(2-methyl-2-(2-oxoacetyl)hydrazinyl)-1-oxopentan-2-yl)amino)-1-oxopentan-2-yl)carbamate (Cbz-Leu-Leu-AAla-COH)

A 0° C. of benzyl ((S)-4-methyl-1-(((S)-4-methyl-1-(2-methylhydrazinyl)-1-oxopentan-2-yl)amino)-1-oxopentan-2-yl)carbamate (50 mg, 0.12 mmol) in DCM (0.5 mL) was treated with a solution of 2-oxoacetyl chloride in DCM (0.5 mL) followed by DIPEA (0.03 mL, 0.18 mmol). The reaction was warmed to RT and stirred 4 h, then diluted with water and extracted with DCM. The organic extracts were washed with sat. NaHCO₃, brine, dried over Na₂SO₄ and concentrated. The crude product was passed through a plug of silica gel (5% MeOH-DCM) and then recrystallized from EtOAc-hexanes to afford the title product as a white solid (6 mg, 11% yield). ¹H NMR (DMSO, 400 MHz) δ: 10.72-10.33 (m, 1H), 8.40-8.02 (m, 1H), 7.50-7.24 (m, 6H), 5.03 (s, 2H), 4.36-4.19 (m, 1H), 4.16-4.01 (m, 1H), 2.95 (s, 3H), 1.73-1.56 (m, 2H), 1.52-1.33 (m, 4H), 0.94-0.79 (m, 12H); ESI MS: found [C₂₄H₃₈N₄O₇+Na]⁺ (methanol hemiacetal).

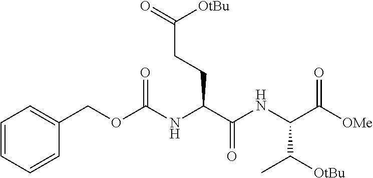 [SMILES: COC(=O)[C@@H](NC(=O)[C@H](CCC(=O)OC(C)(C)C)NC(=O)OCc1ccccc1)C(C)OC(C)(C)C]

### tert-butyl (S)-4-(((benzyloxy)carbonyl)amino)-5-(((2S,3S)-3-(tert-butoxy)-1-methoxy-1-oxobutan-2-yl)amino)-5-oxopentanoate (Cbz-Glu(OtBu)-Thr(OtBu)-OMe)

Z-Glu(OtBu)-OH (1 eq) was dissolved in dry THF and cooled to −20° C. NMM (1 eq) and iBCF (1 eq) were added dropwise and the mixture was allowed to react for 30 min. H-Thr(tBu)-OMe (1 eq) was dissolved in dry THF and cooled to −20° C. NMM (1 eq) were added dropwise and the mixture was allowed to react for 15 min. The two mixtures above were added together. The combined mixture was stirred for 1 h at −20° C. and was allowed to react for 18 h at room temperature. The solvent was evaporated and the residue was treated with EtOAc and H₂O. The organic layer was washed with 1 M HCl, H₂O, salted NaHCO₃, and salted NaCl, dried over Na₂SO₄, filtered and concentrated to give Z-Glu(OtBu)-Thr(tBu)-OMe as a white in 83% yield. ¹H NMR (DMSO-d₆): 0.92 (s, 12H, CH₃CHOC(CH₃)₃), 1.54 (s, 9H, OC(CH₃)₃), 1.87 (m, 1H, CH₂CH₂), 1.91 (m, 1H, CH₂CH₂), 2.27 (t, 2H, CH₂CH₂), 3.62 (s, 3H, OCH₃), 4.18 (m, 2H, CH₃CHO, CHCO), 4.35 (dd, 1H, CHCO), 5.03 (s, 2H, CH₂Ph), 7.32 (s, 5H, Ph), 7.53 (d, 1H, NH), 7.67 (d, 1H, NH). ESI MS: (C₂₆H₄₀N₂NaO₈) Measured 531.2651 m/z, Theoretical 531.2677 m/z

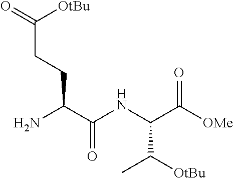 [SMILES: COC(=O)[C@@H](NC(=O)[C@@H](N)CCC(=O)OC(C)(C)C)C(C)OC(C)(C)C]

### tert-butyl (S)-4-amino-5-(((2S,3S)-3-(tert-butoxy)-1-methoxy-1-oxobutan-2-yl)amino)-5-oxopentanoate (H-Glu(OtBu)-Thr(OtBu)-OMe)

Z-Glu(OtBu)-Thr(tBu)-OMe (1 eq) was dissolved in methanol and 10% Pd/C (0.1 eq) and reacted for 18 h under 50 psi hydrogen. Product was filtered through celite and concentrated then purified by column 5% methanol in DCM to obtain H-Glu(OtBu)-Thr(tBu)-OMe as a yellow solid in 28% yield. ¹H NMR (DMSO-d₆): 0.88 (s, 12H, CH₃CHOC(CH₃)₃), 1.39 (s, 9H, OC(CH₃)₃,), 1.58 (m, 1H, CH₂CH₂), 1.88 (m, 1H, CH₂CH₂), 1.97 (s, 2H, NH₂), 2.28 (t, 1H, CH₂CH₂), 3.234 (q, 1H, CH₃CHO), 3.63 (s, 3H, OCH₃), 4.16 (m, 1H, CHCO), 4.30 (dd, 1H, CHCO), 8.03 (d, 1H, NH). ESI MS: (C₁₈H₃₅N₂O₆) Measured 375.2473 m/z, Theoretical 375.2490 m/z

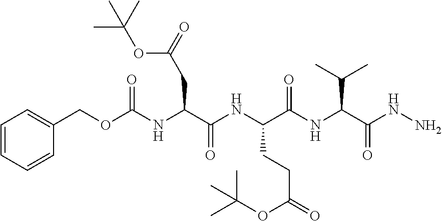 [SMILES: CC(C)[C@H](NC(=O)[C@H](CCC(=O)OC(C)(C)C)NC(=O)[C@H](CC(=O)OC(C)(C)C)NC(=O)OCc1ccccc1)C(=O)NN]

### tert-butyl (S)-4-((S)-2-(((benzyloxy)carbonyl)amino)-4-(tert-butoxy)-4-oxobutanamido)-5-(((S)-1-hydrazinyl-3-methyl-1-oxobutan-2-yl)amino)-5-oxopentanoate (Cbz-Asp(OtBu)-Glu(OtBu)-Val-NHNH₂)

A solution of methyl (5S,8S,11S)-5-(2-(tert-butoxy)-2-oxoethyl)-8-(3-(tert-butoxy)-3-oxopropyl)-11-isopropyl-3,6,9-trioxo-1-phenyl-2-oxa-4,7,10-triazadodecan-12-oate (7.9 g, 12.7 mmol) in) MeOH (80 mL) was treated with hydrazine (2.67 mL, 80 mmol). The reaction was stirred at RT for 18 h, then solvent and excess hydrazine were removed by rotary evaporation to afford the title compound as a white solid. Used for further transformations without further purification (7.9 g, 100%). ¹H NMR (DMSO, 400 MHz) δ: 9.16 (s, 1H), 7.98 (d, 1H), 7.80 (d, 1H), 7.64 (d, 1H), 7.44-7.26 (m, 5H), 5.13-4.95 (m, 2H), 4.46-4.11 (m, 4H), 4.04 (t, 1H), 2.69-2.36 (m, 2H), 2.30-2.14 (m, 2H), 1.96-1.81 (m, 2H), 1.52-1.30 (m, 18H), 0.89-0.76 (m, 6H).

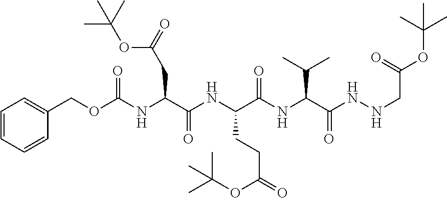 [SMILES: CC(C)[C@H](NC(=O)[C@H](CCC(=O)OC(C)(C)C)NC(=O)[C@H](CC(=O)OC(C)(C)C)NC(=O)OCc1ccccc1)C(=O)NNCC(=O)OC(C)(C)C]

### tert-butyl (5S,8S,1S)-5-(2-(tert-butoxy)-2-oxoethyl)-8-(3-(tert-butoxy)-3-oxopropyl)-11-isopropyl-3,6,9,12-tetraoxo-1-phenyl-2-oxa-4,7,10,13,14-pentaazahexadecan-16-oate (Cbz-Asp(OtBu)-Glu(OtBu)-Val-NHNHCH₂COOtBu)

A −15° C. solution of tert-butyl (S)-4-((S)-2-(((benzyloxy)carbonyl)amino)-4-(tert-butoxy)-4-oxobutanamido)-5-(((S)-1-hydrazinyl-3-methyl-1-oxobutan-2-yl)amino)-5-oxopentanoate (1 g, 1.6 mmol) in DMF (6 mL) was treated with the dropwise addition of t-butylbromoacetate (0.26 mL, 1.76 mmol). The reaction was stirred for 30 min, then warmed to RT and stirred 16 h. Solvent was removed in vacuo, then the residue was purified by silica gel chromatography (4% MeOH-DCM) to afford the title product as a white solid (115 mg, 10%); ¹H NMR (DMSO, 400 MHz) δ: 9.48 (d, 1H), 7.95 (d, 1H), 7.85 (d, 1H), 7.62 (d, 1H), 7.47-7.22 (m, 5H), 5.18-5.11 (m, 1H), 5.10-4.98 (m, 2H), 4.51-4.19 (m, 2H), 4.11-3.99 (m, 1H), 3.41-3.38 (m, 2H), 2.27-2.16 (m, 2H), 1.96-1.69 (m, 5H), 1.46-1.34 (m, 27H), 0.86-0.77 (m, 6H).

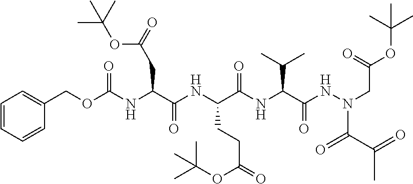 [SMILES: CC(=O)C(=O)N(CC(=O)OC(C)(C)C)NC(=O)[C@@H](NC(=O)[C@H](CCC(=O)OC(C)(C)C)NC(=O)[C@H](CC(=O)OC(C)(C)C)NC(=O)OCc1ccccc1)C(C)C]

### tert-butyl (5S,8S,11S)-5-(2-(tert-butoxy)-2-oxoethyl)-8-(3-(tert-butoxy)-3-oxopropyl)-11-isopropyl-3,6,9,12-tetraoxo-14-(2-oxopropanoyl)-1-phenyl-2-oxa-4,7,10,13,14-pentaazahexadecan-16-oate (Cbz-Asp(OtBu)-Glu(OtBu)-Val-AAsp(OtBu)-COMe)

Prepared following the general procedure for coupling of aza-peptide to pyruvic acid as previously described; (36 mg, 29% yield); ¹H NMR (CDCl₃, 400 MHz) δ: 9.02 (s, 1H), 7.90 (d, 1H), 7.46-7.33 (m, 5H), 6.11-5.99 (m, 1H), 5.22-5.10 (m, 2H), 4.55-4.45 (m, 1H), 4.37-4.25 (m, 2H), 4.24-4.16 (m, 1H), 3.01-2.74 (m, 2H), 2.57-2.39 (m 2H), 2.37 (s, 3H), 2.33-2.24 (m, 1H), 2.19-1.99 (m, 2H), 1.49-1.44 (m, 27H), 0.97-0.92 (m, 6H), ESI MS calc for [C₃₉H₅₉N₅O₁₃+Na]: 828.4002, Found: 828.3984.

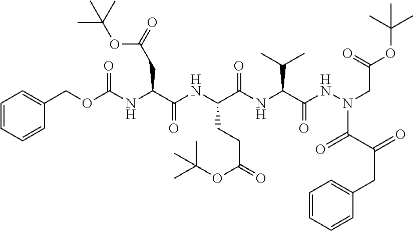 [SMILES: CC(C)[C@H](NC(=O)[C@H](CCC(=O)OC(C)(C)C)NC(=O)[C@H](CC(=O)OC(C)(C)C)NC(=O)OCc1ccccc1)C(=O)NN(CC(=O)OC(C)(C)C)C(=O)C(=O)Cc1ccccc1]

### tert-butyl (5S,8S,11S)-5-(2-(tert-butoxy)-2-oxoethyl)-8-(3-(tert-butoxy)-3-oxopropyl)-11-isopropyl-3,6,9,12-tetraoxo-4-(2-oxo-3-phenylpropanoyl)-1-phenyl-2-oxa-4,710,14-pentaazahexadecan-16-oate (Cbz-Asp(OtBu)-Glu(OtBu)-Val-AAsp(OtBu)-COBn)

Prepared following the general procedure for coupling of aza-peptide to phenylpyruvic acid as previously described: (19 mg, 18% yield); ¹H NMR (DMSO, 400 MHz) δ: 11.07 (s, 1H), 8.00 (d, 1H), 7.93 (d, 1H), 7.60 (d, 1H), 7.40-7.15 (m, 11H), 5.12-4.96 (m, 2H), 4.41-4.28 (m, 2H), 4.21-4.12 (m, 1H), 4.10-3.96 (m, 2H), 2.70-2.57 (m, 1H), 2.47-2.39 (m, 1H), 2.26-2.14 (m, 2H), 1.94-1.80 (m, 2H), 1.77-1.65 (m, 1H), 1.44 (s, 9H), 1.37-1.32 (m, 18H), 0.81-0.75 (m, 6H); ESI MS calc for [C₄₅H₆₃N₅O₁₃+Na]: 904.4315, Found: 904.4282.

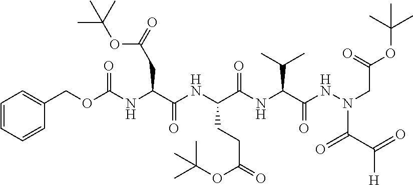 [SMILES: CC(C)[C@H](NC(=O)[C@H](CCC(=O)OC(C)(C)C)NC(=O)[C@H](CC(=O)OC(C)(C)C)NC(=O)OCc1ccccc1)C(=O)NN(CC(=O)OC(C)(C)C)C(=O)C=O]

### tert-butyl (5S,8S,11S)-5-(2-(tert-butoxy)-2-oxoethyl)-8-(3-(tert-butoxy)-3-oxopropyl)-11-isopropyl-3,6,9,12-tetraoxo-14-(2-oxoacetyl)-1-phenyl-2-oxa-4,7,10,13,14-pentaazahexadecan-16-oate (Cbz-Asp(OtBu)-Glu(OtBu)-Val-AAsp(OtBu)-COH)

Prepared following the general procedure for coupling of aza-peptide to glyoxalic acid as previously described. ¹H NMR (DMSO, 400 MHz) δ: 10.68 (s, 1H), 8.01-7.92 (m, 1H), 7.61 (d, 1H), 7.41-7.25 (m, 5H), 5.11-4.98 (m, 2H), 4.42-4.26 (m, 2H), 4.25-4.16 (m, 1H), 4.15-4.02 (m, 2H), 2.70-2.60 (m, 2H), 2.25-2.14 (m 1H), 2.04-1.64 (m, 3H), 1.46-1.36 (m, 27H), 0.93-0.81 (m, 6H); ESI MS calc for [C₃₉H₆₆N₅O₁₄+Na]: 846.4107, Found: 846.4105 (Methanol Hemiacetal).

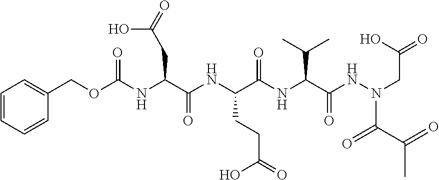 [SMILES: CC(=O)C(=O)N(CC(=O)O)NC(=O)[C@@H](NC(=O)[C@H](CCC(=O)O)NC(=O)[C@H](CC(=O)O)NC(=O)OCc1ccccc1)C(C)C]

### (5S,8S,11S)-8-(2-carboxyethyl)-5-(carboxymethyl)-11-isopropyl-3,6,9,12-tetraoxo-14-(2-oxopropanoyl)-1-phenyl-2-oxa-4,7,10,13,14-pentaazahexadecan-16-oic acid (Cbz-Asp-Glu-Val-AAsp-COMe)

A solution of tert-butyl (5S,8S,11S)-5-(2-(tert-butoxy)-2-oxoethyl)-8-(3-(tert-butoxy)-3-oxopropyl)-11-isopropyl-3,6,9,12-tetraoxo-14-(2-oxopropanoyl)-1-phenyl-2-oxa-4,7,10,13,14-pentaazahexadecan-16-oate (36 mg, 0.045 mmol) in DCM (0.5 mL) was treated with trifluoroacetic acid (0.5 mL). The reaction was stirred 2 h, then concentrated to dryness in vacuo to afford the title product as a white solid (27 mg, 95% yield). ¹H NMR (DMSO, 400 MHz) δ: 11.02 (br s, 1H), 8.17-7.78 (m, 2H), 7.73-7.53 (d, j=8.0 Hz, 1H), 7.42-7.29 (m, 5H), 5.04 (s, 2H), 4.42-4.29 (m, 2H), 4.13 (t, j=6.8 Hz, H), 2.70-2.57 (m, 2H), 2.48-2.43 (m, 1H), 2.29-2.18 (m, 5H), 1.96-1.82 (m, 2H), 0.82 (d, j=6.4 Hz, 6H). ESI MS: found [C₂₇H₃₅N₅O₁₃+Na]⁺660.2116.

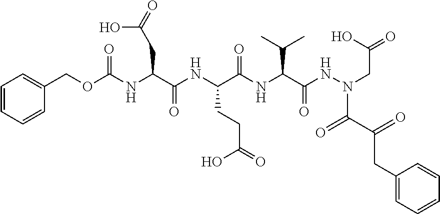 [SMILES: CC(C)[C@H](NC(=O)[C@H](CCC(=O)O)NC(=O)[C@H](CC(=O)O)NC(=O)OCc1ccccc1)C(=O)NN(CC(=O)O)C(=O)C(=O)Cc1ccccc1]

### (5S,8S,11S)-8-(2-carboxyethyl)-5-(carboxymethyl)-11-isopropyl-3,6,9,12-tetraoxo-14-(2-oxo-3-phenylpropanoyl)-1-phenyl-2-oxa-4,7,10,13,14-pentaazahexadecan-16-oic acid (Cbz-Asp-Glu-Val-AAsp-COBn)

tert-butyl (5S,8S,11S)-5-(2-(tert-butoxy)-2-oxoethyl)-8-(3-(tert-butoxy)-3-oxopropyl)-11-isopropyl-3,6,9,12-tetraoxo-14-(2-oxo-3-phenylpropanoyl)-1-phenyl-2-oxa-4,7,10,13,14-pentaazahexadecan-16-oate (18 mg, 0.02 mmol) was dissolved in a 1:1 DCM:trifluoroacetic acid mixture (1.5 mL). The mixture was stirred for 2.5 h, then solvent was removed, and product was dried on high vac to give the title compound as a white solid (13.6 g, 95%). ¹H NMR (DMSO, 400 MHz) δ: 11.06 (s, 1H), 8.02-7.91 (m, 2H), 7.61 (d, j=8 Hz, 1H), 7.42-7.15 (m, 10H), 5.03 (s, 2H), 4.44-4.27 (m, 3H), 4.21-4.12 (m, 2H), 4.06-3.94 (m, 3H), 2.70-2.60 (m, 1H), 2.80-2.18 (m, 2H), 1.95-1.84 (m, 2H), 1.80-1.70 (m, 1H), 0.84-0.70 (m, 6H); ESI MS: calc for: [C₃₃H₃₉N₅O₁₃+Na]⁺736.2437, found 736.2415.

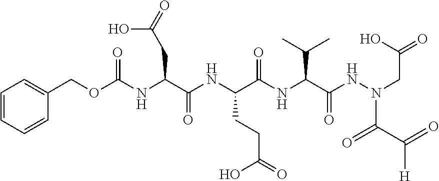 [SMILES: CC(C)[C@H](NC(=O)[C@H](CCC(=O)O)NC(=O)[C@H](CC(=O)O)NC(=O)OCc1ccccc1)C(=O)NN(CC(=O)O)C(=O)C=O]

### (5S,8S,11S)-8-(2-carboxyethyl)-5-(carboxymethyl)-11-isopropyl-3,6,9,12-tetraoxo-14-(2-oxoacetyl)-1-phenyl-2-oxa-4,7,10,13,14-pentaazahexadecan-16-oic acid (Cbz-Asp-Glu-Val-AAsp-COH)

tert-butyl (5S,8S,11S)-5-(2-(tert-butoxy)-2-oxoethyl)-8-(3-(tert-butoxy)-3-oxopropyl)-11-isopropyl-3,6,9,12-tetraoxo-14-(2-oxoacetyl)-1-phenyl-2-oxa-4,7,10,13,14-pentaazahexadecan-16-oate (18 mg, 0.25 mmol) was dissolved in a 1:1 DCM:trifluoroacetic acid mixture (1.5 mL). The mixture was stirred for 2.5 h, then solvent was removed, and product was dried on high vac to give the title compound as a white solid (1.6 mg, 10%). ¹H NMR (CDCl₃, 400 MHz) δ: 7.60 (d, 1H), 7.38-7.27 (m, 6H), 5.14-5.04 (m, 2H), 4.51-4.43 (m, 1H), 4.38-4.29 (m, 1H), 4.19-4.10 (m, 1H), 3.40 (s, 2H), 2.89-2.73 (m, 2H), 2.21-2.10 (m, 2H), 2.03-1.89 (m, 2H), 0.99-0.89 (m, 6H); ESI MS: calc. for [C₂₇H₃₇N₅O₁₄+Na] 678.2235 (Methanol hemiacetal), found: [C₂₇H₃₇N₅O₁₄+Na]⁺678.2236.

### Example 10. Measuring Inhibitor Potency

Kinetic assays were performed on a fluorescent 96-well plate reader with human 20S proteasome. Chymotryptic activity was measured in 20 mM HEPES, 0.5 mM EDTA, 0.037% SDS, pH 7.8 at 37° C., using 0-100 μM Suc-LLVY-AMC as the fluorogenic substrate, and 20 4.42×10⁻⁴ mg/100 μL enzyme.

Triplicates of velocities (fluorescence vs time) were obtained at various concentrations of substrate (10, 20, 50, 100 kM) and aza-peptide inhibitor (0, 25, 50, 100 μM) then converted to specific activity using Lambert-Beer's law and enzyme concentration.

Enzyme specific activities were fit to a competitive inhibition model using GraphPad Prism software for nonlinear fitting.

Results from kinetic assays for compounds tested against the 20S proteasome, caspase-3, and legumain are shown in Table 4, Table 5, and Table 6, respectively.

TABLE 4
20S Proteasome Inhibitors

| PG | P3 | P2 | P1 | Warhead | Ki (μM) |
|---|---|---|---|---|---|
| Z | Leu | Leu | ALeu | COMe | 27 |
| Z | Leu | Leu | ALeu | COBn | 81 |
| Z | Leu | Leu | ALeu | COOEt | weak |
| Z | Leu | Leu | ALeu | COH | 24 |
| Z | Leu | Phe | ALeu | COMe | 47 |
| Z | Leu | Phe | ALeu | COBn | 55 |
| Z | Leu | Phe | ALeu | COH | NA |
| Z | Leu | Leu | AGly | COMe | 29 |
| Z | Leu | Leu | AGly | COH | 35 |
| Z | Leu | Leu | AAla | COMe | 73 |
| Z | Leu | Leu | AAla | COH | 44 |
| Pz | — | Phe | ALeu | COMe | 474 |
| Z | Leu | Leu | Leu | COH | 0.0142^{a} |

“a” is a commercially available inhibitor obtained from Sigma-Aldrich. Its kinetic data is used as a reference.

TABLE 5
Caspase 3 Inhibitors

| PG | P4 | P3 | P2 | P1 | Warhead | IC₅₀ (μM) |
|---|---|---|---|---|---|---|
| Z | Asp | Glu | Val | AAsp | COMe | 0.055 |
| Z | Asp | Glu | Leu | AAsp | COBn | NA |
| Z | Asp | Glu | Leu | AAsp | COH | NA |
| Z | Asp | Glu | Val | Asp | COH | 0.0045^{b} |

^{b}is a commercially available inhibitor obtained from Sigma-Aldrich. Its kinetic data is used as a reference.

TABLE 6
Legumain Inhibitors

| PG | P3 | P2 | P1 | Warhead | Ki (μM) |
|---|---|---|---|---|---|
| Z | Ala | Ala | AAsn | COMe | Weak |
| Z | Ala | Ala | AAsn | COOEt | Weak |
| Z | Ala | Ala | AAsn | COBn | Weak |

Abbreviations in Tables

### PG: Protecting Group

Z: carboxybenzyl
Pz: pyrazinyl
Me: methyl
Et: ethyl
Bn: benzyl
ALeu: aza-leucine
AGly: aza-glycine
AAla: aza-alanine
AAsp: aza-aspartic acid
AAsn: aza-asparagine

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A compound of the formula: 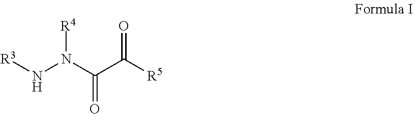
wherein R₃ is selected from the group consisting of M₁, M₂-AA₁, M₂-AA₂-AA₁, and M₂-AA₃-AA₂-AA₁;
M₁ is selected from the group consisting of NH₂—CO—, NH₂—CS—, NH₂—SO₂—, X—NH—CO—, X₂N—CO—, X—NH—CS—, X₂N—CS—, X—NH—SO₂—, X₂N—SO₂—, X—CO—, X—CS—, Y-S02-, Y—O—CO—, Y—O—CS—, phenyl substituted with K, phenyl disubstituted with K, and morpholine-CO—;
M₂ is selected from the group consisting of H, NH₂—CO—, NH₂—CS—, NH₂—SO₂—, X—NH—CO—, X₂N—CO—, X—NH—CS—, X₂N—CS—, X—NH—SO₂—, X₂N—SO₂—, X—CO—, X—CS—, Y—SO₂—, Y—O—CO—, Y—O—CS—, phenyl, phenyl substituted with K, phenyl disubstituted with K, and morpholine-CO—;
X is selected from the group consisting of H, C₁₋₁₀ alkyl, C₃₋₁₅ cyclized alkyl, C₁₋₁₀ fluoroalkyl, C₁₋₁₀ alkyl substituted with J, C₁₋₁₀ fluoroalkyl substituted with J, 1-admantyl, 9-fluorenyl, aryl, heteroaryl, phenyl, phenyl substituted with K, phenyl disubstituted with K, phenyl trisubstituted with K, naphthyl, naphthyl substituted with K, naphthyl disubstituted with K, naphthyl trisubstituted with K, C₁₋₁₀ fluoroalkyl with an attached phenyl group, C₁₋₁₀ alkyl with an attached phenyl group, C₁₋₁₀ alkyl with two attached phenyl groups, C₁₋₁₀ alkyl with an attached phenyl group substituted with K, C₁₋₁₀ alkyl with two attached phenyl groups substituted with K, C₁₋₁₀ alkyl with an attached naphthyl group, C₁₋₁₀ alkyl with an attached naphthyl group substituted with K, C₁₋₁₀ alkyl with an attached phenoxy group, biotinyl, and C₁₋₁₀ alkyl with an attached phenoxy group substituted with K on the phenoxy group;
Y is selected from the group consisting of C₁₋₁₀ alkyl, C₃₋₁₅ cyclized alkyl, C₁₋₁₀ fluoroalkyl, C₁₋₁₀ alkyl substituted with J, C₁₋₁₀ fluoroalkyl substituted with J, 1-admantyl, 9-fluorenyl, phenyl, phenyl substituted with K, phenyl disubstituted with K, phenyl trisubstituted with K, naphthyl, naphthyl substituted with K, naphthyl disubstituted with K, naphthyl trisubstituted with K, C₁₋₁₀ fluoroalkyl with an attached phenyl group, C₁₋₁₀ alkyl with an attached phenyl group, C₁₋₁₀ alkyl with two attached phenyl groups, C₁₋₁₀ alkyl with an attached phenyl group substituted with K, C₁₋₁₀ alkyl with two attached phenyl groups substituted with K, C₁₋₁₀ alkyl with an attached naphthyl group, C₁₋₁₀ alkyl with an attached naphthyl group substituted with K, C₁₋₁₀ alkyl with an attached phenoxy group, biotinyl, and C₁₋₁₀ alkyl with an attached phenoxy group substituted with K on the phenoxy group;
J is selected from the group consisting of halogen, CO₂H, OH, CN, NO₂, NH₂, C₁₋₁₀ alkoxy, C₁₋₁₀ alkylamino, C₂₋₁₂ dialkylamino, C₁₋₁₀ alkyl-O—CO—, C₁₋₁₀ alkyl-O—CO—NH—, and C₁₋₁₀ alkyl-S—;
K is selected from the group consisting of halogen, C₁₋₁₀ alkyl, C₁₋₁₀ perfluoroalkyl, C₁₋₁₀ alkoxy, phenoxy, NO₂, CN, OH, CO₂H, amino, C₁₋₁₀ alkylamino, C₂₋₁₂ dialkylamino, C₁₋₁₀ acyl, and C₁₋₁₀ alkoxy-CO—, and C₁₋₁₀ alkyl-S—;
AA₁, AA₂, and AA₃ are side chain blocked or unblocked amino acids with the L configuration, D configuration, or no chirality at the α-carbon independently selected from the group consisting of alanine, valine, leucine, isoleucine, proline, methionine, methionine sulfoxide, phenylalanine, tryptophan, glycine, serine, threonine, cysteine, tyrosine, asparagine, glutamine, aspartic acid, glutamic acid, lysine, arginine, histidine, phenylglycine, beta-alanine, norleucine, norvaline, alpha-aminobutanoic acid, epsilon-aminocaproic acid, citrulline, hydroxyproline, ornithine, homoarginine, sarcosine, indoline 2-carboxylic acid, 2-azetidinecarboxylic acid, pipecolinic acid (2-piperidine carboxylic acid), O-methylserine, O-ethylserine, S-methylcysteine, S-ethylcysteine, S-benzylcysteine, NH₂—CH(CH₂CHEt₂)-CO₂H, alpha-aminoheptanoic acid, NH₂—CH(CH₂₋₁-naphthyl)-CO₂H, NH₂—CH(CH₂-2-naphthyl)-CO₂H, NH₂—CH(CH₂-cyclohexyl)-CO₂H, NH₂—CH(CH₂-cyclopentyl)-CO₂H, NH₂—CH(CH₂-cyclobutyl)-CO₂H, NH₂—CH(CH₂-cyclopropyl)-CO₂H, trifluoroleucine, 4-fluorophenylalanine, lysine substituted on the epsilon nitrogen with a biotinyl group, and hexafluoroleucine;
R₄ is selected from the group consisting of hydrogen, C₁₋₁₀ alkyl, C₁₋₁₀ alkyl substituted with Q, C₁₋₁₀ alkyl substituted with phenyl, C₁₋₁₀ alkyl with an attached phenyl substituted with K, C₁₋₁₀ alkyl substituted with naphthyl, C₁₋₁₀ alkyl with an attached naphthyl substituted with K, phenyl, phenyl substituted with K, naphthyl, naphthyl substituted with K, C₁₋₁₀ alkyl substituted with CONH₂, C₁₋₁₀ alkyl substituted with CONHR₆, C₁₋₁₀ alkyl substituted with CO₂H, C₁₋₁₀ alkyl substituted with CO₂R₆, CH₂CH₂SCH₃, CH₂₋₃-indolyl, CH₂-2-thienyl, CH₂-2-furyl, CH₂-3-furyl, CH₂-2-imidazyl, C₁₋₁₀ alkyl substituted with G, C₁₋₁₀ alkyl with an attached phenyl substituted with G, C₁₋₁₀ alkyl with an attached naphthyl substituted with G, phenyl substituted with G, and naphthyl substituted with G;
R₆ is selected from the group consisting of C₁₋₁₀ alkyl and C₁₋₁₀ alkyl substituted with phenyl;
Q is selected independently from the group consisting of C₁₋₁₀ alkoxy, C₁₋₁₀ alkyl-S—, C₁₋₁₀ alkoxy substituted with phenyl, and C₁₋₁₀ alkyl-S— substituted with phenyl;
G is selected independently from the group consisting of amidino (—C(═NH)NH₂), guanidino (—NHC(═NH)NH₂), isothiureido (—S—C(═NH)NH₂), amino, C₁₋₆ alkylamino, C₂₋₁₂ dialkylamino, and imidazyl;
R₅ is selected independently from the group consisting of hydrogen, R₇, NHRs, NRsR9, and -AA₄-T;
R₇ is selected independently from the group consisting of C₁₋₁₀ alkyl, C₃₋₁₅ cyclized alkyl, C₁₋₁₀ alkyl with a phenyl group attached to the C₁₋₁₀ alkyl, C₃₋₁₅ cyclized alkyl with an attached phenyl group, C₁₋₁₀ alkyl with an attached phenyl group substituted with K, C₁₋₁₀ alkyl with an attached phenyl group disubstituted with K, C₁₋₁₀ alkyl with an attached phenyl group trisubstituted with K, C₃₋₁₅ cyclized alkyl with an attached phenyl group substituted with K, C₁₋₁₀ alkyl with a naphthyl group attached to the C₁₋₁₀ alkyl, C₃₋₁₅ cyclized alkyl with an attached naphthyl group, C₁₋₁₀ alkyl with an attached naphthyl group substituted with K, C₁₋₁₀ alkyl with an attached naphthyl group disubstituted with K, C₁₋₁₀ alkyl with an attached naphthyl group trisubstituted with K, and C₃₋₁₅ cyclized alkyl with an attached naphthyl group substituted with K;
T is selected independently from the group consisting of OH, OR₁₀, NHR₁₁, and NR₁₀R₁₁1;
AA₄ is a side chain blocked or unblocked amino acid with the L configuration, D configuration, or no chirality at the α-carbon selected from the group consisting of alanine, valine, leucine, isoleucine, proline, methionine, methionine sulfoxide, phenylalanine, tryptophan, glycine, serine, threonine, cysteine, tyrosine, asparagine, glutamine, aspartic acid, glutamic acid, lysine, arginine, histidine, phenylglycine, beta-alanine, norleucine, norvaline, alpha-aminobutanoic acid, epsilon-aminocaproic acid, citrulline, hydroxyproline, ornithine, homoarginine, sarcosine, indoline 2-carboxylic acid, 2-azetidinecarboxylic acid, pipecolinic acid (2-piperidine carboxylic acid), O-methylserine, O-ethylserine, S-methylcysteine, S-ethylcysteine, S-benzylcysteine, NH₂—CH(CH₂CHEt₂)-CO₂H, alpha-aminoheptanoic acid, NH₂—CH(CH₂-1-naphthyl)-CO₂H, NH₂—CH(CH₂-2-naphthyl)-CO₂H, NH₂—CH(CH₂-cyclohexyl)-CO₂H, NH₂—CH(CH₂-cyclopentyl)-CO₂H, NH₂—CH(CH₂-cyclobutyl)-CO₂H, NH₂—CH(CH₂-cyclopropyl)-CO₂H, trifluoroleucine, 4-fluorophenylalanine, lysine substituted on the epsilon nitrogen with a biotinyl group, and hexafluoroleucine;
R₈ and R₉ are selected independently from the group consisting of H, C₁₋₁₀ alkyl, C₃₋₂₀ cyclized alkyl, C₁₋₁₀ alkyl with a phenyl group attached to the C₁₋₁₀ alkyl, C₁₋₁₀ alkyl with two phenyl groups attached to the C₁₋₁₀ alkyl, C₃₋₂₀ cyclized alkyl with an attached phenyl group, phenyl, phenyl substituted with K, C₁₋₁₀ alkyl with an attached phenyl group substituted with K, C₁₋₁₀ alkyl with an attached phenyl group disubstituted with K, C₁₋₁₀ alkyl with an attached phenyl group trisubstituted with K, C₁₋₁₀ alkyl with two phenyl groups attached to the C₁₋₁₀ alkyl and substituted with K on the phenyl group, C₁₋₁₀ alkyl with two phenyl groups attached to the C₁₋₁₀ alkyl and disubstituted with K on the phenyl groups, C₃₋₂₀ cyclized alkyl with an attached phenyl group substituted with K, C₁₋₁₀ alkyl with a morpholine [—N(CH₂CH₂)O] ring attached through nitrogen to the alkyl, C₁₋₁₀ alkyl with a piperidine ring attached through nitrogen to the alkyl, C₁₋₁₀ alkyl with a pyrrolidine ring attached through nitrogen to the alkyl, C₁₋₂₀ alkyl with an OH group attached to the alkyl, —CH₂CH₂CH₂OCH₃, C₁₋₁₀ alkyl with an attached 4-pyridyl group, C₁₋₁₀ alkyl with an attached 3-pyridyl group, C₁₋₁₀ alkyl with an attached 2-pyridyl group, C₁₋₁₀ alkyl with an attached cyclohexyl group, —NH—CH₂CH₂-(4-hydroxyphenyl), —NH—CH₂CH₂-(3-indolyl), C₁₋₁₀ alkyl with an attached 2-furyl group, C₁₋₁₀ alkyl with an attached 3-furyl group, and C₁₋₅ alkyl with an attached phenyl and a hydroxyl attached to the C₁₋₅ alkyl;
R₁₀ and R₁₁ are selected independently from the group consisting of H, C₁₋₁₀ alkyl, phenyl, nitrophenyl, and C₁₋₁₀ alkyl substituted with phenyl;
or a pharmaceutically acceptable salt, derivative, hydrate or solvate thereof.

2. The compound of claim 1, having the formula: 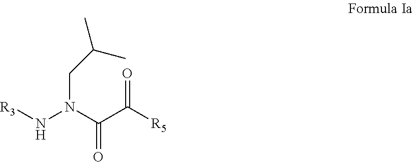
wherein R₃ and R₅ are defined according to claim 1;
or a pharmaceutically acceptable salt, derivative, hydrate or solvate thereof.

3. The compound of claim 1, having the formula: 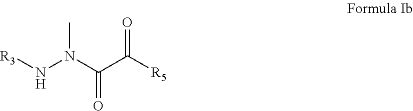
wherein R₃ and R₅ are defined according to claim 1;
or a pharmaceutically acceptable salt, derivative, hydrate or solvate thereof.

4. The compound of claim 1, having the formula: 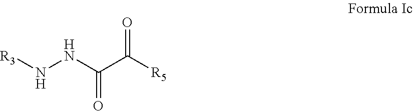
wherein R₃ and R₅ are defined according to claim 1;
or a pharmaceutically acceptable salt, derivative, hydrate or solvate thereof.

5. The compound of claim 1, having the formula: 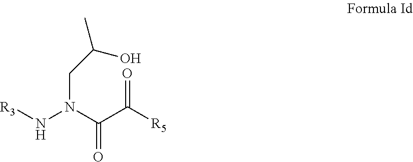
wherein R₃ and R₅ are defined according to claim 1;
or a pharmaceutically acceptable salt, derivative, hydrate or solvate thereof.

6. The compound of claim 1, having the formula: 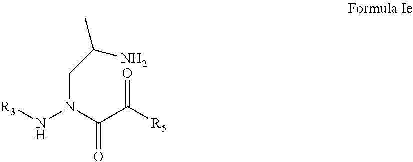
wherein R₃ and R₅ are defined according to claim 1;
or a pharmaceutically acceptable salt, derivative, hydrate or solvate thereof.

7. The compound of claim 1, having the formula: 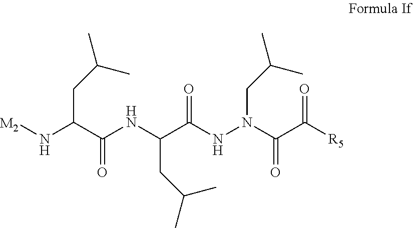
wherein M₂ and R₅ are defined according to claim 1;
or a pharmaceutically acceptable salt, derivative, hydrate or solvate thereof.

8. The compound of claim 1, wherein R₃ is M₂-AA₂-AA₁.

9. The compound of claim 1, wherein R₃ is M₂-AA₃-AA₂-AA₁.

10. The compound of claim 1, wherein M₂ is Y—O—CO—, wherein Y is benzyl.

11. The compound of claim 1, wherein R₅ is selected independently from the group consisting of hydrogen, C₁₋₁₀ alkyl, and C₁₋₁₀ alkyl with a phenyl group attached to the C₁₋₁₀ alkyl.

12. The compound of claim 11, wherein R₅ is hydrogen.

13. The compound of claim 11, wherein R₅ is methyl.

14. The compound of claim 11, wherein R₅ is benzyl.

15. The compound of claim 1, selected from the group consisting of:
Cbz-Leu-Leu-ALeu-COH;
Cbz-Leu-Leu-ALeu-COMe;
Cbz-Leu-Leu-ALeu-COBn;
Cbz-Leu-Phe-ALeu-COH;
Cbz-Leu-Phe-ALeu-COMe;
Cbz-Leu-Phe-ALeu-COBn;
Cbz-Leu-Leu-AGly-COH;
Cbz-Leu-Leu-AGly-COMe;
Cbz-Leu-Leu-AAla-COH; and
Cbz-Leu-Leu-AAla-COMe;
or a pharmaceutically acceptable salt, derivative, hydrate or solvate thereof.

16. The compound of claim 1, selected from the group consisting of:
Cbz-Asp-Glu-Val-AAsp-COMe; and
Cbz-Asp-Glu-Leu-AAsp-COBn;
or a pharmaceutically acceptable salt, derivative, hydrate or solvate thereof.

17. The compound of claim 1, selected from the group consisting of:
Cbz-Ala-Ala-AAsn-COH;
Cbz-Ala-Ala-AAsn-COMe; and
Cbz-Ala-Ala-AAsn-COBn;
or a pharmaceutically acceptable salt, derivative, hydrate or solvate thereof.

18. A method of inhibiting a protease comprising contacting the protease with a compound of claim 1.

19.-23. (canceled)

24. A method of treating a neurodegenerative disease in a subject in need thereof, comprising administering to the subject a therapeutically effective amount of a compound of claim 1.

25. (canceled)

26. A method of treating a cancer in a subject in need thereof, comprising administering to the subject a therapeutically effective amount of a compound of claim 1.
